(19) United States
(12) Patent Application Publication
(10) Pub. No.: US 2009/0117559 A1
(43) Pub. Date: May 7, 2009

(54) Methods for Determining Probability of an Adverse or Favorable Reaction to a Niacin Receptor Agonist

(76) Inventors: Chen W. Liaw, San Diego, CA (US); Martha Kanemitsu-Parks, Del Mar, CA (US); Jeremy G. Richman, San Diego, CA (US); Dominique Maciejewski-Lenoir, San Diego, CA (US); Daniel T. Connolly, Solana Beach, CA (US)

Correspondence Address: FISH & RICHARDSON P.C., P.O. BOX 1022, MINNEAPOLIS, MN, 55440-1022, US

(21) Appl. No.: 11/990,240

(22) PCT Filed: Aug. 9, 2006
(86) PCT No.: PCT/US2006/031032

§ 371 Date: Feb. 8, 2008

Related U.S. Application Data
(60) Provisional application No. 60/707,372, filed on Aug. 10, 2005.

(51) Int. Cl. C12Q 1/68

(52) U.S. Cl. 435/6

(57) ABSTRACT

The present invention relates generally to a GPR109A niacin receptor. The present invention relates more particularly to assessing a GPR109A polymorphism in an individual, wherein the GPR109A polymorphism is indicative of the subject's risk for an adverse reaction to the administration of a GPR109A receptor agonist, wherein the adverse reaction is associated with stimulation of MAP kinase activity by the GPR109A receptor agonist. More specifically, the present invention relates to assessing a GPR109A polymorphism in an individual and determining the level of risk for the subject for experiencing an adverse reaction, wherein the subject's GPR109A zygosity is predictive of the risk for a cutaneous flushing response that can be experienced following administration of a GPR109A receptor agonist.

### FIELD OF THE INVENTION

The present invention relates generally to assays for determining an individual's probability for a condition, adverse or favorable, associated with a functional niacin receptor-mediated signal response. For example, the present invention relates to determining if an individual has an elevated or reduced probability for an adverse reaction to the administration of a niacin receptor agonist.

### BACKGROUND OF THE INVENTION

Atherosclerosis is a process whereby deposits of fatty substances, cholesterol and other substances build up in the inner lining of an artery. This buildup is called plaque. Plaques that rupture cause blood clots to form that can block blood flow to the heart (heart attack) or the brain (stroke). Heart attack is the number one cause of death for both men and women in the United States and stroke is the number three cause of death [see, for example, Nature Medicine, Special Focus on Atherosclerosis, (2002) 8:1209-1262]. Abnormally high levels of circulating lipids are a major predisposing factor in development of atherosclerosis. Elevated levels of low density lipoprotein (LDL) cholesterol, elevated levels of free fatty acids, elevated levels of triglycerides, or low levels of high density lipoprotein (HDL) cholesterol are, independently, risk factors for atherosclerosis and associated pathologies.

Niacin (nicotinic acid, pyridine-3-carboxylic acid, vitamin B3) is a water-soluble vitamin required by the human body for health, growth and reproduction. Recently, niacin has been shown to be an agonist of the G protein-coupled receptor GPR109A (GenBank Accession No. NM_{—}177551), known also as HM74A. The GPR109A niacin receptor is encoded by a single exon and couples to Gi. An agonist to the niacin receptor lowers the level of intracellular cAMP. (See, e.g., U.S. Pat. No. 6,902,902.) More recently, (D)-β-hydroxybutyrate has been shown to be an endogenous agonist of GPR109A [Taggart et al., J Biol Chem (2005) 280:26649-26652]. GPR109A has been shown to be polymorphic at several amino acid positions, for example an arginine/cysteine polymorphism at amino acid position 311 (R311C) and a methionine/isoleucine polymorphism at amino acid position 317 (M317I) [Zellner et al., Hum Mutat (2005) 25:18-21].

Niacin is one of the oldest used drugs for the treatment of lipid-associated disorders. It is a valuable drug in that it favorably affects virtually all of the lipid parameters listed above [Goodman and Gilman's Pharmacological Basis of Therapeutics, editors Harmon J G and Limbird L E, Chapter 36, Mahley R W and Bersot T P (2001) pages 971-1002]. The benefits of niacin in the treatment or prevention of atherosclerotic cardiovascular disease have been documented in six major clinical trials [Guyton J R (1998) Am J Cardiol 82:18U-23U]. Structure and synthesis of analogs or derivatives of niacin are discussed throughout the Merck Index, An Encyclopedia of Chemicals, Drugs, and Biologicals, Tenth Edition (1983).

Unfortunately, the doses of niacin required to alter serum lipid levels can be quite large and at these dosages adverse side effects are frequent. Side effects can include gastrointestinal disturbances, liver toxicity, and disruption of glucose metabolism and uric acid levels. However, the most frequent and prominent side effect of niacin therapy is cutaneous vasodilation, also called flushing (or cutaneous flushing), characterized by cutaneous itching, tingling and warmth. Flushing is believed to be caused by the niacin-induced release of prostaglandin D2 (PGD2) in the skin. Although the flushing associated with niacin administration is generally harmless, it is sufficiently unpleasant that patient compliance is markedly reduced. Often, 30-40% of patients cease taking niacin treatment within days after initiating therapy.

It has been observed that the skin flush response to niacin is diminished in many individuals with schizophrenia. Schizophrenia is a common psychiatric disease which affects about 1% of the population. A diminished skin flush response to niacin is among the most widely replicated peripheral physiological abnormalities in schizophrenia [Messamore, Prostaglandins, Leukotrienes and Essential Fatty Acids (2003) 69:413-419]. Recently, an impaired flush response to niacin has been shown to be associated with acute first-episode schizophrenia [Smesny et al., Prostaglandins, Leukotrienes and Essential Fatty Acids (2005) 72:393-402]. This abnormal skin flush response to niacin has been suggested to be a marker of the deficiency in essential fatty acids (EFAs) documented to be present in many patients with schizophrenia. The blunted skin flush response to niacin has been suggested to have diagnostic value for schizophrenia (see, e.g., WO 97/45145 and Puri B K et al., Int J Clin Pract (2001) 55:368-370). The ability to distinguish an impaired niacin-mediated flushing response consistent with a deficiency in EFAs from a more common impaired niacin-mediated flushing response unrelated to a deficiency in EFAs would be useful.

Generally, being able to determine whether or not an individual is at risk of flushing on administration of niacin or an analog thereof would be highly beneficial. Thus, there exists a need for an assay whereby an individual's probability for niacin-induced flushing or other adverse or favorable effects of a niacin receptor agonist can be rapidly and easily determined. The present invention satisfies this need and provides related advantages as well.

### SUMMARY OF THE INVENTION

Applicants have shown that niacin receptor (GPR109A) agonists that activate mitogen-activated protein kinase (MAP kinase; MAPK) to a lesser extent than niacin cause less flushing than niacin in vivo (see FIG. 1).

The present invention relates generally to a GPR109A niacin receptor. The present invention relates more particularly to assessing a GPR109A polymorphism in an individual, wherein the GPR109A polymorphism is informative as to the individual's probability for a condition, adverse or favorable, associated with a functional niacin receptor-mediated signal response. For example, the present invention relates to assessing a GPR109A polymorphism in an individual, wherein the GPR109A polymorphism is informative as to the individual's probability for a favorable reaction to the administration of a GPR109A receptor agonist, for example, elevation of high density lipoprotein (HDL), atheroma regression or reverse cholesterol transport. Also, for example, the present invention relates to assessing a GPR109A polymorphism in an individual, wherein the GPR109A polymorphism is informative as to the individual's probability for an adverse reaction to the administration of a GPR109A receptor agonist, for example, where the adverse reaction is associated with stimulation of MAP kinase activity by the GPR109A receptor agonist. More specifically, the present invention relates to assessing a GPR109A polymorphism in an individual and determining the level of probability for the individual for experiencing an adverse reaction, wherein the individual's GPR109A zygosity is predictive of the probability for a cutaneous flushing response that can be experienced following administration of a GPR109A receptor agonist.

In a first aspect, the invention provides a method of determining an individual's probability for a condition associated with a functional niacin receptor-mediated signal response, comprising obtaining a GPR109A receptor nucleic acid sequence or a GPR109A receptor amino acid sequence for the individual; identifying within said nucleic acid sequence or said amino acid sequence a nucleotide at a position corresponding to nucleotide position 951 of SEQ ID NO:1 or an amino acid at a position corresponding to amino acid position 317 of SEQ ID NO:2; and assigning a level of probability to the individual for the condition associated with a functional niacin receptor-mediated signal response.

In one embodiment, the nucleotide at the position corresponding to nucleotide position 951 of SEQ ID NO:1 or the amino acid at the position corresponding to amino acid position 317 of SEQ ID NO:2 is identified to be homozygous or heterozygous in the individual. In another embodiment, an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with a functional niacin receptor-mediated signal response.

In one embodiment, homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with a functional niacin receptor-mediated signal response. In another embodiment, homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with a functional niacin receptor-mediated signal response.

In one embodiment, said method is for use in predicting an individual's probability for the condition associated with a functional niacin receptor-mediated signal response in a therapy for a lipid disorder, wherein said therapy comprises administration of an amount of niacin or the niacin analog. In another embodiment, the amount of niacin or the niacin analog is a therapeutically effective amount. In a further embodiment, said GPR109A receptor nucleic acid sequence or said GPR109A amino acid sequence is obtained from a database.

In one embodiment, the condition associated with a functional niacin receptor-mediated signal response is cutaneous flushing. In another embodiment, the condition associated with a functional niacin receptor-mediated signal response is high density lipoprotein (HDL) elevation. In a further embodiment, the condition associated with a functional niacin receptor-mediated signal response is atheroma regression. In a yet further embodiment, the condition associated with a functional niacin receptor-mediated signal response is reverse cholesterol transport.

In a second aspect, the invention provides a method of determining a level of probability for an individual for a condition associated with stimulation of MAP kinase activity by niacin or a niacin analog, comprising obtaining a biological sample from the individual; identifying within said biological sample a nucleotide at a position corresponding to nucleotide position 951 of SEQ ID NO:1 or an amino acid at a position corresponding to amino acid position 317 of SEQ ID NO:2; and assigning the level of probability to the individual for the condition associated with stimulation of MAP kinase activity by niacin or the niacin analog.

In one embodiment, the nucleotide at the position corresponding to nucleotide position 951 of SEQ ID NO:1 or the amino acid at the position corresponding to amino acid position 317 of SEQ ID NO:2 is identified to be homozygous or heterozygous in the individual. In a further embodiment, an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with stimulation of MAP kinase activity by niacin or the niacin analog.

In one embodiment, the homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with stimulation of MAP kinase activity by niacin or the niacin analog. In another embodiment, the homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with stimulation of MAP kinase activity by niacin or the niacin analog.

In another embodiment, a further step is added wherein a portion of the GPR109A gene spanning nucleotides 949 to 951 of SEQ ID NO:1 is amplified prior to the identifying step. In one embodiment, the portion of the GPR109A gene spanning nucleotides 949 to 951 of SEQ ID NO:1 is amplified by polymerase chain reaction (PCR). In another embodiment, the identifying is performed by a method selected from the group consisting of a hybridization assay, a sequencing assay, a microsequencing assay, a MALDI-TOF assay, and an allele-specific amplification assay. In a further embodiment, the identifying is performed by an antibody-based assay.

In one embodiment, the method is for use in predicting an individual's probability for the condition associated with the stimulation of MAP kinase activity by niacin or a niacin analog in a therapy for a lipid disorder, wherein said therapy comprises administration of an amount of niacin or the niacin analog. In another embodiment, the amount of niacin or the niacin analog is a therapeutically effective amount. In a further embodiment, the condition associated with stimulation of MAP kinase activity by niacin or the niacin analog is cutaneous flushing.

In a third aspect, the invention provides a method of determining a level of probability for an individual for a condition associated with stimulation of MAP kinase activity by niacin or a niacin analog, comprising obtaining a GPR109A receptor nucleic acid sequence or a GPR109A receptor amino acid sequence for the individual; identifying within the nucleic acid sequence or the amino acid sequence a nucleotide at a position corresponding to nucleotide position 951 of SEQ ID NO:1 or an amino acid at a position corresponding to amino acid position 317 of SEQ ID NO:2; and assigning the level of probability to the individual for the condition associated with stimulation of MAP kinase activity by niacin or the niacin analog.

In one embodiment, the nucleotide at the position corresponding to nucleotide position 951 of SEQ ID NO:1 or the amino acid at the position corresponding to amino acid position 317 of SEQ ID NO:2 is identified to be homozygous or heterozygous in the individual. In another embodiment, an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with stimulation of MAP kinase activity by niacin or the niacin analog. In a further embodiment, the homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with stimulation of MAP kinase activity by niacin or the niacin analog. In another embodiment, homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with stimulation of MAP kinase activity by niacin or the niacin analog.

In a further embodiment, the method is for use in predicting an individual's probability for the condition associated with stimulation of MAP kinase activity by niacin or a niacin analog in a therapy for a lipid disorder, wherein said therapy comprises administration of an amount of niacin or the niacin analog. In one embodiment, the amount of niacin or the niacin analog is a therapeutically effective amount. In another embodiment, the GPR109A receptor nucleic acid sequence or said GPR109A amino acid sequence is obtained from a database. In a further embodiment, the condition associated with stimulation of MAP kinase activity by niacin or the niacin analog is cutaneous flushing.

In a fourth aspect, the invention is directed to any one of the applicable methods provided above, wherein the method is for use in selection of a therapy comprising administration of an amount of niacin or a niacin analog for a lipid disorder, wherein said therapy is selected so as to ameliorate a condition associated with stimulation of MAP kinase activity by niacin or the niacin analog. In one embodiment, the amount of niacin or the niacin analog is a therapeutically effective amount. In another embodiment, the condition associated with stimulation of MAP kinase activity by niacin or the niacin analog is cutaneous flushing.

In a fifth aspect, the invention is directed to any one of the applicable methods provided above, wherein the method is for use in determining a suitability or an unsuitability of the individual for inclusion in a clinical trial for assessing an efficacy of an amount of a GPR109A receptor agonist for treating a lipid disorder without or with less of a condition associated with stimulation of MAP kinase activity by niacin or a niacin analog. In one embodiment, a zygosity of the individual is indicative of the individual being unsuitable for inclusion in the clinical trial, wherein the zygosity is selected from the group consisting of: homozygosity for an adenine at a nucleotide position corresponding to nucleotide position 951 of SEQ ID NO:1 or for an isoleucine at an amino acid position corresponding to amino acid position 317 of SEQ ID NO:2; and homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2. In another embodiment, a zygosity of the individual is indicative of the individual being suitable for inclusion in the clinical trial, wherein the zygosity is homozygosity for a guanine at a nucleotide position corresponding to nucleotide position 951 of SEQ ID NO:1 or for a methionine at an amino acid position corresponding to amino acid position 317 of SEQ ID NO:2. In a further embodiment, the condition associated with stimulation of MAP kinase activity by niacin or the niacin analog is cutaneous flushing.

In a sixth aspect, the invention is directed to any one of the applicable methods provided above, wherein the method is for use in determining a suitability or an unsuitability of the individual for inclusion in a clinical trial for assessing an efficacy of a compound for ameliorating a condition associated with stimulation of MAP kinase activity by niacin or the niacin analog. In one embodiment, the compound is an inhibitor of prostaglandin D2 activity. In another embodiment, a zygosity of the individual is indicative of the individual being unsuitable for inclusion in the clinical trial, wherein the zygosity is selected from the group consisting of homozygosity for an adenine at a nucleotide position corresponding to nucleotide position 951 of SEQ ID NO:1 or for an isoleucine at an amino acid position corresponding to amino acid position 317 of SEQ ID NO:2; and homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2. In another embodiment, a zygosity of the individual is indicative of the individual being suitable for inclusion in the clinical trial, wherein the zygosity is homozygosity for a guanine at a nucleotide position corresponding to nucleotide position 951 of SEQ ID NO:1 or for a methionine at an amino acid position corresponding to amino acid position 317 of SEQ ID NO:2. In a further embodiment, the condition associated with stimulation of MAP kinase activity by niacin or the niacin analog is cutaneous flushing.

In a seventh aspect, the invention is directed to any one of the applicable methods provided above, wherein the method is for use in classifying the individual according to a level of probability for a condition associated with stimulation of MAP kinase activity by niacin or a niacin analog. In one embodiment, the condition associated with stimulation of MAP kinase activity by niacin or the niacin analog is cutaneous flushing.

In an eighth aspect, the invention provides a method of using a GPR109A receptor zygosity of an individual for determining a suitability or an unsuitability of the individual for inclusion in a clinical trial, wherein said zygosity is indicative of a level of probability for the individual for a condition associated with the stimulation of MAP kinase activity by niacin or a niacin analog. In one embodiment, the GPR109A receptor zygosity is selected from the group consisting of homozygosity for an adenine at a nucleotide position corresponding to nucleotide position 951 of SEQ ID NO:1 or for an isoleucine at an amino acid position corresponding to amino acid position 317 of SEQ ID NO:2; heterozygosity for an adenine at a nucleotide position corresponding to nucleotide position 951 of SEQ ID NO:1 or for an isoleucine at an amino acid position corresponding to amino acid position 317 of SEQ ID NO:2; homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2; and homozygosity for a guanine at a nucleotide position corresponding to nucleotide position 951 of SEQ ID NO:1 or for a methionine at an amino acid position corresponding to amino acid position 317 of SEQ ID NO:2. In a further embodiment, the clinical trial is selected from the group consisting of: a clinical trial for assessing the efficacy of a GPR109A receptor agonist for treating a lipid disorder without or with less of a condition associated with stimulation of MAP kinase activity by niacin or a niacin analog; a clinical trial for assessing the efficacy of a compound in ameliorating a condition associated with stimulation of MAP kinase activity by niacin or the niacin analog; and a clinical trial for assessing the efficacy of a compound for treating schizophrenia.

In a ninth aspect, the invention provides a method of determining a level of probability for a condition associated with stimulation of MAP kinase activity by niacin or a niacin analog for an individual having a GPR109A receptor zygosity comprising the steps of: identifying a clinical outcome for each of a plurality of patients in a clinical trial comprising a therapy, wherein the therapy comprises administration of an amount of niacin or a niacin analog, and wherein the clinical outcome is exhibiting or not exhibiting the condition associated with the stimulation of MAP kinase activity by niacin or the niacin analog; obtaining or identifying the GPR109A receptor zygosity for each of said plurality of patients in the clinical trial; associating the clinical outcome and the GRP109A receptor zygosity for each of said plurality of patients; and analyzing the associated clinical outcomes and GPR109A receptor zygosities so as to allow assignment of a level of probability for the condition associated with stimulation of MAP kinase activity by niacin or the niacin analog for the individual having the GPR109A receptor zygosity. In one embodiment, the amount of niacin or the niacin analog is a therapeutically effective amount. In another embodiment, the GPR109A receptor zygosity is selected from the group consisting of: homozygous for an adenine at a nucleotide position corresponding to nucleotide position 951 of SEQ ID NO:1 or for an isoleucine at an amino acid position corresponding to amino acid position 317 of SEQ ID NO:2; heterozygous for an adenine at a nucleotide position corresponding to nucleotide position 951 of SEQ ID NO:1 or for an isoleucine at an amino acid position corresponding to amino acid position 317 of SEQ ID NO:2; and homozygous for an G at a nucleotide position corresponding to nucleotide position 951 of SEQ ID NO:1 or for a methionine at an amino acid position corresponding to amino acid position 317 of SEQ ID NO:2. In a further embodiment, the analyzing comprises the steps of: segmenting or not segmenting the clinical outcomes on the basis of the GPR109A receptor zygosity so as to thereby make a segmented group and an unsegmented group; and comparing the clinical outcomes for the segmented group with the clinical outcomes for the unsegmented group. In one embodiment, the condition associated with the stimulation of MAP kinase activity by niacin or the niacin analog is cutaneous flushing.

In a tenth aspect, the invention provides a method of determining a level of probability for an individual for a condition associated with stimulation of MAP kinase activity by niacin or a niacin analog, said method comprising the steps of: (a) obtaining a GPR109A receptor nucleic acid sequence or a GPR109A receptor amino acid sequence for the individual; (b) identifying within said GPR109A receptor nucleic acid sequence a nucleotide polymorphism compared to SEQ ID NO:1, or within said GPR109A receptor amino acid sequence an amino acid polymorphism compared to SEQ ID NO:2; and (c) assessing the ability of said GPR109A receptor nucleic acid sequence containing said nucleotide polymorphism or GPR109A receptor amino acid sequence containing said amino acid polymorphism to affect MAP kinase activation mediated by niacin, wherein a blunted MAP kinase activation compared to the MAP kinase activation of a GPR109A receptor containing SEQ ID NO:2 is associated with a decreased level of probability to the individual for the condition associated with stimulation of MAP kinase activity by niacin or the niacin analog. In one embodiment, the condition associated with the stimulation of MAP kinase activity by niacin or the niacin analog is cutaneous flushing.

These and other aspects and features of the invention will be readily apparent to the ordinarily skilled artisan upon reviewing the disclosure provided herein.

### BRIEF DESCRIPTION OF THE DRAWINGS

FIG. 1 shows a graph of MAP kinase activation by several niacin receptor agonists in Chinese hamster ovary (CHO) cells expressing recombinant wild type human GPR109A. Long arrows indicate compounds that have been shown to flush in mice and short arrows indicate compounds that do not flush in mice. The horizontal line indicates two standard deviations below niacin. In FIG. 1, the recombinant wildtype GPR109A receptor is encoded by the nucleotide sequence of SEQ ID NO:1 and has the amino acid sequence of SEQ ID NO: 2.

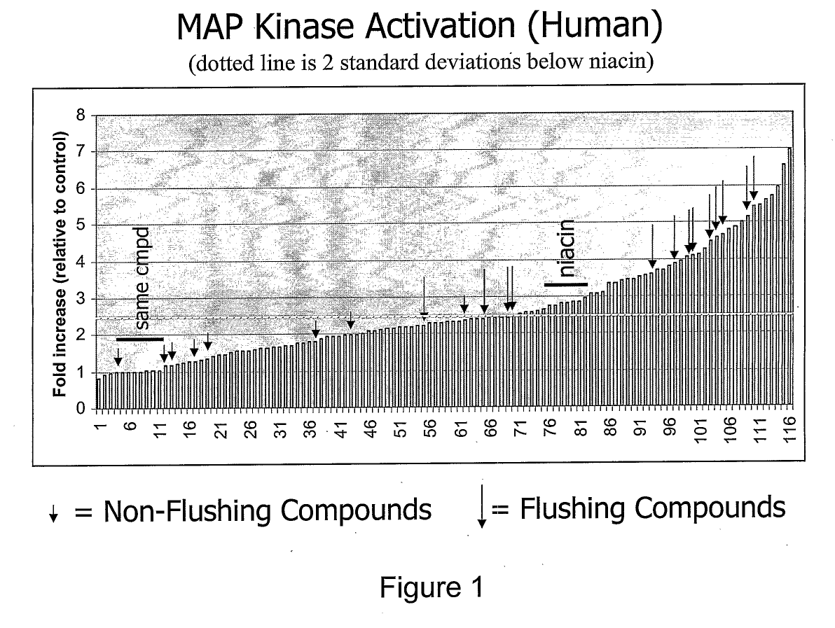

FIG. 2 shows the effects of the C311 and I317 amino acid polymorphisms on niacin-mediated stimulation of MAP kinase activation.

FIG. 3 shows the effects of the C311 and I317 amino acid polymorphisms on niacin-mediated reduction in intracellular cAMP.

FIG. 4 shows the effects of the C311 and I317 amino acid polymorphisms on niacin-mediated reduction in intracellular cAMP by highlighting the EC₅₀ of GPR109A wild-type from FIG. 3.

FIG. 5 shows the effects of the C311 and I317 amino acid polymorphisms on niacin-mediated reduction in intracellular cAMP by highlighting the EC₅₀ of GPR109A C311 from FIG. 3.

FIG. 6 shows the effects of the C311 and I317 amino acid polymorphisms on niacin-mediated reduction in intracellular cAMP by highlighting the EC₅₀ of GPR109A I317 from FIG. 3.

FIG. 7 shows haplotype and zygosity frequencies of M317 and I317 for GPR109A.

### DEFINITIONS

Nucleotide abbreviations as used herein are A (adenine), G (guanine), C (cytosine), and T (thymine).

The term “polymorphism”, as used herein, refers to a difference in the nucleotide or amino acid sequence of a given nucleotide or amino acid region as compared to a nucleotide or amino acid sequence in the corresponding region of another individual of the same species. Preferably, the species is human. A polymorphism is generally defined in relation to a “reference” sequence. In the subject application, “reference” sequence and “wild type” sequence are used interchangeably. Nucleotide polymorphisms include single nucleotide differences, differences in sequence of more than one nucleotide, and single or multiple nucleotide insertions, inversions, substitutions, and deletions. Amino acid polymorphisms include single amino acid differences, differences in sequence of more than one amino acid, and single or multiple amino acid insertions, substitutions, and deletions.

For example, the term “polymorphic GPR109A” or “polymorphic niacin receptor” refers to a polynucleotide or polypeptide derived from a GPR109A gene, which polynucleotide or polypeptide comprises one or more polymorphisms when compared to a “reference” GPR109A polynucleotide or polypeptide sequence. In the subject application, the reference (“wild type”; “GPR109A wt”) GPR109A polynucleotide is a mammalian, for instance a human GPR109A coding region having the nucleotide sequence of SEQ ID NO:1, and the reference (“wild type”; “GPR109A wt”) GPR109A polypeptide is mammalian, for instance a human GPR109A encoded by SEQ ID NO:1 (provided by SEQ ID NO:2). “GPR109A” and “niacin receptor” are used interchangeably herein.

A polymorphism in a mammalian (e.g., human) polymorphic GPR109A receptor can be associated with a decreased niacin-mediated stimulation of MAP kinase activity relative to the mammalian (e.g., human) wildtype GPR109A receptor of the subject invention, or can be associated with similar or increased niacin-mediated stimulation of MAP kinase activity relative to the mammalian wildtype (e.g., human) GPR109A receptor.

When determining or detecting a polymorphism in a GPR109A receptor, particularly a single nucleotide polymorphism, several means are known in the art, such as those described herein below. For instance, in one embodiment, determining a polymorphism in a GPR109A receptor means identifying a codon substitution at position 311 of the amino acid of SEQ ID NO: 2, for instance, wherein an arginine (R) is replaced with a cysteine (C). Additionally, in another embodiment, determining means identifying the presence or the absence of a codon substitution at position 317 of the amino acid of SEQ ID NO: 2, for instance, wherein a methionine (M) is replaced with an isoleucine (I). In further embodiments, determining means identifying a single nucleotide polymorphism at position 931 of SEQ ID NO:1, whereby a C is replaced with a T; or identifying a single nucleotide polymorphism at position 951 of SEQ ID NO:1, whereby a guanine is replaced with an A, T, or C. It is to be noted that isoleucine can be coded for by three different codons: ATT, ATC, and ATA, therefore any single nucleotide polymorphism at position 951, whereby an A, T, or C replaces a G, results in the substitution of a methionine with an isoleucine. Said determining can be done by any means well known in the art, as described in greater detail herein below.

The terms “polynucleotide” and “nucleic acid molecule” are used interchangeably herein to refer to polymeric forms of nucleotides of any length. The polynucleotides can contain deoxyribonucleotides, ribonucleotides, and/or their analogs. Nucleotides can have any three-dimensional structure, and can perform any function, known or unknown. The term polynucleotide includes single-, double-stranded and triple helical molecules. Oligonucleotide generally refers to polynucleotides of between about 3 and about 100 nucleotides of single- or double-stranded DNA. However, for the purposes of this disclosure, there is no upper limit to the length of an oligonucleotide. Oligonucleotides are also known as oligomers or oligos and can be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule can also comprise modified nucleic acid molecules, such as methylated nucleic acid molecules and nucleic acid molecule analogs. Analogs of purines and pyrimidines are known in the art. Nucleic acids can be naturally occurring, e.g. DNA or RNA, or can be synthetic analogs, as known in the art. Such analogs can be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α-anomer of deoxyribose can be used, where the base is inverted with respect to the natural β-anomer. The 2′-OH of the ribose sugar can be altered to form 2′-O— methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

A codon refers to a set of three consecutive nucleotides in a strand of DNA or RNA that provides the genetic information to code for a specific amino acid which will be incorporated into a protein chain or serve as a termination signal.

The terms “polypeptide” and “protein”, used interchangebly herein, refer to a polymeric form of amino acids of any length, which can include encoded and non-encoded amino acids, chemically or biochemically modified or derivatized amino acids, and polypeptides having modified peptide backbones.

In the broadest sense, as used herein, the term “blunted MAP kinase activity” refers to MAP kinase enzymatic activity that is lower than the MAP kinase enzymatic activity associated with niacin-mediated stimulation of the wildtype GPR109A receptor of the subject invention, as determined by methods well known in the art. In the same way, increased MAP kinase activity refers to MAP kinase enzymatic activity that is higher than the MAP kinase enzymatic activity associated with niacin-mediated stimulation of the wildtype GPR109A receptor of the subject invention.

A condition associated with normal or elevated niacin-stimulated MAP kinase activity is a condition that is symptomatic of normal or elevated niacin-stimulated MAP kinase activity, such as cutaneous flushing. Normal or elevated MAP kinase enzymatic activity refers to niacin-stimulated MAP kinase activity that is similar or increased relative to that exhibited by the wild type GPR109A receptor of the subject invention. A representative type of a condition associated with normal or elevated MAP kinase activity is flushing or a condition related thereto.

As used herein, the terms “flushing” and “cutaneous flushing” are used interchangeably and refer to a condition (also referred to herein as a symptomatic condition or a disorder) which is associated with niacin-mediated stimulation of MAP kinase activity. Specifically, the term flushing means a detectable cutaneous vasodilation reaction. For example, flushing can be caused by administration of a niacin receptor agonist such as niacin or a niacin analog. Niacin-induced flushing is believed to be mediated through prostaglandins such as prostaglandin D2 (PGD2). A flushing reaction is characterized by redness of the skin and can also include other symptoms, for example, cutaneous itching, tingling, a feeling of warmth, or headache. The flushing reaction can occur anywhere on the skin, for example, on the face, neck or trunk, and can occur in one location or at more than one location. In humans, the flushing reaction can last from several minutes to a several hours. Generally, in humans a flushing reaction caused by oral administration of sufficient doses of niacin or a niacin analog can last anywhere from 20 minutes to 8 hours or more. In a mouse or rat, the flushing reaction usually peaks at about 3 minutes post administration of niacin (by injection) and declines significantly after about 30 minutes.

Flushing can be assessed in the individual. For example, in humans one can use anecdotal evidence by asking the individual to describe their response to a niacin receptor agonist and/or by observing the individual. Several methods can be used to detect and quantify flushing. For example, flushing can be visually detected and quantified. One method for detecting and quantifying flushing is by Laser Doppler, for example using a Pirimed PimII Laser Dopler. In addition, surveys of individuals can be taken to assess flushing and the severity of symptoms that can be associated with flushing such as tingling or a feeling of warmth. Another method for detecting and quantifying flushing can include measurement of the level of prostaglandin D2 (PGD2) or prostaglandin F2 (PGF2; a stable metabolite of prostaglandin D2) in a biological sample from an individual such as blood or urine. In addition, for example, the level of PGD-M, the major urinary metabolite of PGD2 can be measured from the urine of subjects. Assays for measuring prostaglandin levels are commercially available, for example, an enzyme immunoassay for PGD2 is available from Cayman Chemical (Ann Arbor, Mich.).

The amount of niacin or a niacin analog required to produce a detectable flushing reaction depends on several variables, for example, the formulation of the compound and the individual subject. In particular, the amount of niacin or a niacin analog required to produce a detectable flushing reaction can be dependent on, for example, the body weight of the individual, genetic makeup of the individual or general health of the individual. Amounts of niacin or a niacin analog that can cause a flushing reaction in a human can be less than those required to lower the amount of atherosclerosis associated serum lipids and can include, for example, at least 175 mg per day, at least 200 mg per day, at least 250 mg per day, at least 500 mg per day, at least 750 mg per day, at least 1 g per day, at least 1.5 g per day, at least 2 g per day, at least 2.5 g per day, at least 3 g per day, at least 3.5 g per day, at least 4 g per day, at least 4.5 g per day, at least 5 g per day, at least 5.5 g per day, at least 6 g per day, at least 6.5 g per day, at least 7 g per day, at least 7.5 g per day, at least 8 g per day, or more. For example, 500 mg to 2 g or more per day of niacin can cause a flushing reaction in most humans.

As used herein, “niacin” means nicotinic acid which has the following chemical formula:

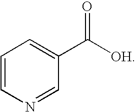

The term niacin also includes pharmaceutically acceptable salts and solvates of niacin which have similar properties to the free acid form of niacin. As understood by one skilled in the art, niacin can be formulated with other compounds such that its pharmacologic properties are modified. For example, niacin can be formulated as an immediate release (IR) form or as an extended or sustained release (SR) form depending on other compounds that are added to the niacin.

As used herein, “niacin analog” means a compound structurally or functionally related to niacin which has a similar MAP kinase activation profile and flushing effect as niacin. Such niacin analogs will be apparent to those of skill in the art. Several structural analogs of niacin are known in the art. Structural analogues of niacin can contain at least one functional acidic group, such as carboxyl, tetrazolyl, and the like. Structural analogues of niacin can also contain at least one nitrogen ring atom, such as the nitrogen present in pyridinyl, pyrazolyl, isoxazolyl, and the like. Additionally, structural analogues of niacin can contain at least one functional acidic group and at least one nitrogen ring atom. These groups can include pro-drug groups that are transformed in vivo to yield the functional acidic group or ring nitrogen, for example, by hydrolysis in blood. A thorough discussion is provided in T. Higuchi and V. Stella, “Pro-drugs as Novel Delivery Systems,” Vol. 14 of the A.C.S. Symposium Series, and in “Bioreversible Carriers in Drug Design,” ed. Edward B. Roche, American Pharmaceutical Association and Pergamon Press, 1987, both of which are hereby incorporated by reference.

A niacin analog can be functionally related to niacin, for example, a niacin analog can have a function of niacin such as specifically binding to the niacin receptor or initiating an intracellular signal in response to binding at the niacin receptor. For example, a niacin analog can be a niacin receptor agonist. Several analogs or derivatives of niacin are known in the art and can be found, for example, in Merck Index, An Encyclopedia of Chemicals, Drugs, and Biologicals, Tenth Edition (1983). As described above for niacin, niacin analogs can be formulated in different ways to modify their pharmacologic properties.

A “GPR109A receptor” as referred to herein is used interchangeably with a “niacin receptor” and means a mammalian, for instance, a human niacin receptor.

A “GPR109A receptor agonist” is material, for example niacin or a niacin analog, which activates an intracellular response in the cell when it binds to or otherwise interacts with the niacin receptor. It will be appreciated that a niacin receptor agonist can be other than niacin or a niacin analog.

In general, a receptor agonist is material, for example, a ligand or compound, which activates an intracellular response when it binds or otherwise interacts with a receptor. An intracellular response can be, for example, enhancement of GTP binding to membranes or modulation of the level of a second messenger such as cAMP. An agonist can also be material not previously known to activate the intracellular response when it binds to the receptor (for example, to enhance GTPγS binding to membranes). Agonist as used herein, and unless explicitly stated otherwise, encompasses full agonists and partial agonists. A partial agonist is material, for example, a ligand or compound, which activates an intracellular response when it binds to the receptor but to a lesser degree or extent than a full agonist

A niacin receptor partial agonist is material that activates an intracellular response when it binds to a niacin receptor but to a lesser degree than does niacin, which is a full agonist at the niacin receptor. Technically, the term partial agonist is a relative term because a partial agonist generates a partial response compared to a full agonist. Since new compounds are being discovered with time, the full agonist can change and a formerly full agonist can become a partial agonist.

For clarity, a niacin receptor partial agonist as used herein is compared to niacin as the full agonist. A niacin receptor partial agonist has a detectably lesser degree of activation of an intracellular response compared to the niacin, i.e. a niacin receptor partial agonist elicits less than a maximal response. Thus, a niacin receptor partial agonist has less efficacy than niacin. For example, a niacin receptor partial agonist has 90% or less efficacy compared to niacin, 85% or less efficacy compared to niacin, 80% or less efficacy compared to niacin, 75% or less efficacy compared to niacin, 70% or less efficacy compared to niacin, 65% or less efficacy compared to niacin, 60% or less efficacy compared to niacin, 55% or less efficacy compared to niacin, 50% or less efficacy compared to niacin, 45% or less efficacy compared to niacin, 40% or less efficacy compared to niacin, 35% or less efficacy compared to niacin, 30% or less efficacy compared to niacin, 25% or less efficacy compared to niacin, 20% or less efficacy compared to niacin, 15% or less efficacy compared to niacin, or 10% efficacy compared to niacin. For example, a niacin receptor partial agonist can have 10% to 90% efficacy compared to niacin, 20% to 80% efficacy compared to niacin, 30% to 70% efficacy compared to niacin, 40% to 60% efficacy compared to niacin, or 45% to 55% efficacy compared to niacin. Efficacy, is the magnitude of the measured response and representative methods of the subject invention involve assessing the efficacy of one or more GPR109A receptor agonist. Efficacy is different from potency which is the amount of compound it takes to elicit a defined response. Therefore, a niacin receptor partial agonist can be more, less, or equally potent when compared to an agonist, antagonist, or inverse agonist.

A niacin receptor partial agonist can be determined using assays well known in the art. For example, a niacin receptor partial agonist can be determined using a cAMP assay.

A niacin receptor specifically binds to niacin. The term specifically binds is intended to mean the polypeptide or protein will have an affinity for a target compound, such as niacin, that is measurably higher than its affinity for an un-related compound.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term biological sample encompasses a clinical sample, and also includes cells in culture, cell supernatants, cell lysates, serum, plasma, biological fluid, and tissue samples. In one embodiment, the sample is collected by the individual. For example, an individual can collect a swap of tissue from the inside of the cheek for use as a nucleic acid sample. As known in the art, many types of samples can be used for the extraction of nucleic acids.

“Reduced” means a decrease in a measurable quantity or a particular activity and is used synonymously with the terms “decreased”, “diminishing”, “lowering”, and “lessening.” In reference to probability for an adverse reaction, such as an adverse condition associated with stimulation of MAP kinase activity by niacin or a niacin analog, an individual having reduced probability for an adverse reaction is intended herein to mean that the subject is more likely to have a reduced adverse reaction. A reduced adverse reaction can be, for example, a decrease in the severity of the adverse reaction and/or fewer adverse reaction events than would otherwise occur (a decrease in the incidence of the adverse reaction). More specifically in reference to a reduced flushing reaction, a reduced flushing reaction can be, for example, a decrease in the severity of flushing and/or fewer flushing events than would otherwise occur (a decrease in the incidence of flushing). For example, the severity and/or incidence of flushing can be decreased at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In addition, flushing can be decreased 100% or eliminated such that no significant flushing is detectable. In one embodiment, the intensity of flushing is decreased at least about 80%. In another embodiment, the decrease in flushing is a complete reduction or elimination of flushing.

The term “level of probability” or “level of risk” refers to the probability that a condition, adverse or favorable, associated with a functional niacin receptor-mediated signal response will occur in an individual. In reference to a “level of probability” for an individual for a favorable reaction associated with a functional niacin receptor-mediated signal response (such as HDL elevation, atheroma regression or reverse cholesterol transport), the probability level can be determined by assessing the GPR109A allele, zygosity, or both, but usually both GPR109A allele and zygosity, of the individual and assessing the “level of probability” for the individual relative to the probability level in a known general population or a known GPR109A subpopulation segmented according to a GPR109A allele or zygosity. In reference to a “level of probability” or “level of risk” for an individual for an adverse reaction such as a condition associated with the stimulation of MAP kinase activity as a result of administration of niacin or a niacin analog (for example, cutaneous flushing), said probability level or risk level can be determined by assessing the GPR109A allele, zygosity, or both, but usually both GPR109A allele and zygosity, of the individual and assessing the “level of probability” or “level of risk” for the individual relative to the probability or risk in a known general population or a known GPR109A subpopulation segmented according to a GPR109A allele or zygosity.

For instance, and not to be limited hereby, a representative example of the incidence of a flushing response with respect to nicotinic acid therapy in a general population has been reported in Shepherd et al., Current Medical Research and Opinion (2005) 21:665-682. Therein, 70% to about 80% of subjects in an unsegmented (e.g., general) population were reported to experience flushing in response to nicotinic acid therapy. Accordingly, in this instance, a “reduced probability” or “reduced risk” for a condition associated with the stimulation of MAP kinase activity would, therefore, constitute a probability or risk that is lower than about 70-80% of that of the general population. Furthermore, an increased or elevated probability or risk would be a probability or risk that is greater than about 70%-80% of that of the general population. For example, in this instance, were a segmented homozygous I317 subpopulation (e.g., a subset) to be considered, the probability or risk of an individual in this subpopulation would be less than that for the general population, that is less than about 70-80%, and would therefore be a reduced probability or risk. On the other hand, if a segmented homozygous M317 subpopulation (e.g., subset) were to be considered, the probability or risk for an individual in the subpopulation would be greater than that for the general population, that is greater than about 70%-80%, and would therefore constitute an elevated probability or risk.

Any assay to assess MAP kinase activity can be used in accordance with the methods of the invention. For example, a substrate activity assay such as an assay using mylein basic protein, which is a substrate for MAP kinase, can be used in the methods of the invention. Additionally, an antibody based assay can be used to determine MAP kinase activity. Such assays are well known in the art and include, for example, Western blot, ELISA, immunoprecipitation, fluorescent polarization assay (FPA), Biacore assay and the like. In one embodiment, the assay used to determine MAP kinase activity is an ELISA. In one embodiment, the assay used to determine MAP kinase activity in the methods of the invention uses the human niacin receptor.

As understood by one skilled in the art, antibodies used in such assays bind specifically to their target, such as MAP kinase. The term binds specifically, in the context of antibody binding, refers to high avidity and/or high affinity binding of an antibody to a specific polypeptide i.e., epitope of a MAP kinase polypeptide. Antibody binding to an epitope on a MAP kinase polypeptide is stronger than binding of the same antibody to any other epitope, particularly those which can be present in molecules in association with, or in the same sample, as the specific polypeptide of interest, e.g., so that by adjusting binding conditions the antibody binds almost exclusively to the specific MAP kinase epitope and not to any other epitopes or other polypeptides.

Antibodies which bind specifically to a MAP kinase polypeptide can be capable of binding other polypeptides at a weak, yet detectable, level (e.g., 10% or less of the binding shown to the polypeptide of interest). Such weak binding, or background binding, is readily discernible from the specific antibody binding to the compound or polypeptide of interest, e.g. by use of appropriate controls. In general, antibodies which bind to a specific MAP kinase polypeptide with a binding affinity of 10⁷ moles/liter or more, preferably 10⁸ moles/liter or more are said to bind specifically to the MAP kinase polypeptide. In general, an antibody with a binding affinity of 106 moles/liter or less is not useful in that it will not bind an antigen at a detectable level using conventional methodology currently used. Methods for detecting or measuring antibody binding are well known in the art.

A detectably labeled antibody refers to an antibody (or antibody fragment that retains binding specificity for a MAP kinase polypeptide or epitope) having an attached detectable label. The detectable label is normally attached by chemical conjugation, but where the label is a polypeptide, it could alternatively be attached by genetic engineering techniques. Methods for production of detectably labeled proteins are well known in the art. Detectable labels can be selected from a variety of such labels known in the art including, but not limited to, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin)), methods for labeling antibodies, and methods for using labeled antibodies are well known in the art (see, for example, Harlow and Lane, eds. (Antibodies: A Laboratory Manual (1988) Cold Spring Harbor Laboratory Press, Cold Spring Harbor, N.Y.)).

As used herein the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder. Treatment, as used in this context, covers any treatment of a symptomatic condition, such as an adverse reaction in a mammal, particularly in a human, and includes: (a) diagnosing and then preventing the adverse reaction from occurring in an individual which can be predisposed to the reaction but has not yet been diagnosed as having it; (b) inhibiting the adverse reaction, i.e., arresting its development; and (c) relieving the adverse reaction, i.e., causing regression of the reaction. The term “therapeutically effective amount,” in this context, therefore, means an amount that is effective in treating a particular disorder; that is an amount that is effective for reducing the severity of one or more symptoms associated with the particular disorder for which treatment is sought (e.g., an amount that is effective for lowering the amount of circulating lipids). The term “ameliorate,” as used for instance in the amelioration of a particular condition means to make one or more symptoms of the condition at least more tolerable, if not better. The term ameliorate does not necessarily mean an increase in toleration of all symptoms associated with a disorder and does not necessarily mean a complete reduction in the severity of one or more symptoms associated with a disorder.

Similarly, the term “preventing” means prevention of the occurrence or onset of one or more symptoms associated with a particular adverse reaction and does not necessarily mean the complete prevention of an adverse reaction. The methods of the invention can be used in the treatment or prevention of a niacin-responsive disorder including, for example, the flushing associated with niacin-mediated stimulation of MAP kinase activity, as described herein.

The term “administration” means the delivery of a therapeutic or pharmaceutical formulation and can be by any route well known in the art, for instance, oral, rectal, nasal, topical application (including buccal and sub-lingual), vaginal or parenteral (including intramuscular, sub-cutaneous and intravenous), inhalation, insufflation delivery or the like. Accordingly, compounds to be administered can be pharmaceutically or therapeutically formulated together with a conventional adjuvant, carrier, or diluent, in a unit dosage form, and in such form can be delivered as solids, such as tablets or filled capsules, or liquids such as solutions, suspensions, emulsions, elixirs, gels or capsules filled with the same, all for oral delivery, in the form of suppositories for rectal administration; in the form of liquids, gels, lotions or ointments for topical administration, or in the form of sterile injectable solutions for parenteral (including subcutaneous) use. Such pharmaceutical or therapeutic compositions and unit dosage forms thereof can comprise conventional ingredients in conventional proportions, with or without additional active compounds or principles, and such unit dosage forms can contain any suitable therapeutically effective amount of the active ingredient commensurate with the intended daily dosage range to be employed.

The terms “individual” refers to a mammal, including, but not limited to, murines, simians, humans, mammalian farm animals, mammalian sport animals, and mammalian pets. The term individual also includes individuals who are patients. In one embodiment, an individual is a human.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citation herein by Applicant of a publication, patent, or published patent application is not an admission by Applicant of said publication, patent, or published patent application as prior art. The disclosures of the publications, patents and patent applications cited herein by Applicant are herein incorporated by reference in their entireties into the present disclosure.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a GPR109A receptor modulator” includes a plurality of such GPR109A receptor modulators and reference to “the GPR109A receptor” includes reference to one or more GPR109A receptors and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### DETAILED DESCRIPTION OF THE INVENTION

The present invention relates generally to assessing a GPR109A polymorphism in an individual and to assessing a level of probability for the individual for a condition associated with a functional niacin receptor-mediated signal response. For example, the invention relates to assessing a GPR109A polymorphism in an individual and assessing a level of probability for the individual for cutaneous flushing, HDL elevation, atheroma regression, or reverse cholesterol transport. Some conditions associated with a functional niacin receptor-mediated signal response are not advantageous to the individual, for example, cutaneous flushing, while other conditions associated with a functional niacin receptor-mediated signal response are advantageous to the individual, for example, HDL elevation, atheroma regression, or reverse cholesterol transport. Specifically, the present invention relates to determining the level of probability for an individual of experiencing a condition associated with a functional niacin receptor-mediated signal response by assessing the GPR109A zygosity of the individual (e.g., determining if the subject has a GPR109A polymorphism, specifically an I317 GPR109A polymorphism), wherein the level of probability for any individual can be determined by assessing the individual's GPR109A zygosity and assessing the “level of probability” for the individual relative to the probability in a known general population or a known GPR109A subpopulation segmented according to a GPR109A allele or zygosity. More specifically, the present invention relates to assessing a GPR109A polymorphism in an individual which is informative as to the individual's probability of experiencing a favorable effect of niacin receptor-mediated signaling (such as HDL elevation, atheroma regression or reverse cholesterol transport) or an adverse effect of niacin receptor-mediated signaling (such as flushing).

In addition, the present invention relates generally to assessing a GPR109A polymorphism in an individual and to assessing the level of probability (e.g., susceptibility) of an individual to an adverse reaction to stimulation of the GPR109A receptor (e.g., by administration of an agonist). Specifically, the present invention relates to determining the level of probability for an individual of experiencing an adverse reaction to the administration of a niacin receptor agonist by assessing the GPR109A zygosity of the individual (e.g., determining if the subject has a GPR109A polymorphism, specifically an I317 GPR109A polymorphism), wherein the level of probability for any individual can be determined by assessing the individual's GPR109A zygosity and assessing the “level of probability” for the individual relative to the probability in a known general population or a known GPR109A subpopulation segmented according to a GPR109A allele or zygosity. More specifically, the present invention relates to assessing a GPR109A polymorphism in an individual which is informative as to the individual's susceptibility to the flushing that can be experienced in response to administration of a niacin receptor agonist (e.g., determining if an individual has a reduced probability or risk of flushing).

The inventors have discovered that the ability to distinguish between various amino acid polymorphisms of the GPR109A G protein-coupled receptor is useful for determining the level of probability for an individual for a favorable or adverse condition associated with a functional niacin receptor-mediated signal response. For example, the inventors have discovered that the ability to distinguish between various amino acid polymorphisms of the GPR109A G protein-coupled receptor is useful for determining the level of probability for an individual (e.g., if an individual has reduced probability) for an adverse flushing reaction in response to administration of a niacin receptor agonist. Specifically, and without being held to theory, the inventors have discovered that the I317 amino acid polymorphism of the GPR109A receptor leads to reduced agonist-mediated stimulation of MAP kinase activity compared with that of wildtype GPR109A receptor. As reduced niacin receptor agonist-mediated stimulation of MAP kinase activity is associated with reduced cutaneous flushing, an individual with the I317 amino acid polymorphism is less likely to exhibit flushing on administration of a niacin receptor agonist than is an individual not having the I317 amino acid polymorphism (e.g., an individual having wildtype GPR109A amino acid sequence). Accordingly, as will be described in greater detail below, an individual with the I317 amino acid polymorphism would have a reduced level of probability of suffering from an adverse flushing side effect when compared to an individual with the wildtype genotype M317 GPR109A polymorphism.

Accordingly, the present invention provides assays for determining the presence or absence of a GPR109A polymorphism useful for predicting the probability of a flushing reaction to administration of niacin or other GPR109A receptor agonist and for assessing the level of probability for an individual for a condition associated with the stimulation of MAP kinase activity (for instance, by the administration of niacin or a niacin analog). The invention also finds use in, for example, rational drug therapy (e.g., to facilitate selection of a therapy for an individual based on the GPR109A zygosity or genotype) and in design of clinical trials involving niacin receptor modulators. For instance, the presence or absence of a GPR109A polymorphism can be determinative of inclusion or exclusion into a clinical trial.

The invention will now be described in more detail.

### GPR109A Polymorphisms

In general, the invention involves determining whether an individual carries a polymorphism of the GPR109A receptor and, based on that determination, assessing the individual's level of probability of experiencing a condition associated with a functional niacin-mediated signal response. For example, the invention involves determining whether an individual carries a polymorphism of the GPR109A receptor, and based on that determination, assessing the individual's level of probability of experiencing a favorable or an adverse reaction associated with niacin receptor agonist-mediated stimulation of a signaling pathway, for example, stimulation of MAP kinase activity, calcium flux, or enhanced cAMP signaling via a Gs pathway (for example, using isoproterenol to stimulate the Gs pathway via β2 adrenergic receptors). For example, an individual who carries a GPR109A nucleic acid polymorphism encoding an amino acid that leads to a reduced niacin-mediated signal response, such as niacin-mediated stimulation of MAP kinase activity, has a reduced level of probability for experiencing a condition associated with a niacin-mediated signal response. For example, an individual who carries a GPR109A nucleic acid polymorphism encoding an amino acid that leads to a reduced niacin-mediated signal response, such as reduced MAP kinase activity, has a reduced level of probability for experiencing a condition associated with a niacin-mediated signal response, such as cutaneous flushing. In addition, for example, an individual who carries a GPR109A nucleic acid polymorphism encoding an amino acid that leads to a reduced niacin-mediated signal response, such as reduced MAP kinase activity, has a reduced level of probability for experiencing a favorable condition associated with a niacin-mediated signal response, such as HDL elevation, atheroma regression, or reverse cholesterol transport. An individual who carries a GPR109A nucleic acid polymorphism encoding an amino acid that does not lead to reduced niacin-mediated stimulation of a niacin-mediated signal response such as MAP kinase activity (e.g., the wildtype genotype) does not have a reduced probability for a condition associated with a niacin-mediated signal response.

Several naturally occurring amino acid polymorphisms of the GPR109A receptor have been described [Zellner et al., Hum Mutat (2005) 25:18-21]. Two such amino acid polymorphisms, GPR109A C311 (“C311”) and GPR109A I317 (“I317”), have been characterized by the present inventors. Both amino acid polymorphisms derive from the wildtype gene by a single nucleotide polymorphism (SNP). Amino acid polymorphisms of the subject invention are detectable at more than one level, including but not limited to genomic DNA, mRNA, cDNA, and GPR109A protein.

Any given subject can be homozygous or heterozygous for a given GPR109A polymorphism, or homozygous or heterozygous for wildtype GPR109A. Accordingly, with reference to GPR109A receptor zygosity, the term “zygosity” includes homozygosity and heterozygosity of a particular nucleotide at a particular position of the GPR109A wildtype sequence of SEQ ID NO:1, as explained in more detail below. For instance, the presence of a guanine or an adenine at the nucleotide position corresponding to nucleotide position 951 of SEQ ID NO:1, wherein, homozygosity would be, for example, a G/G or an A/A and heterozygosity would be a G/A or an A/G at position 951. Additionally, the presence of a C or a T at the nucleotide position corresponding to nucleotide position 931 of SEQ ID NO:1, wherein, for homozygosity would either be a C/C or a T/T and heterozygosity would be a C/T or a T/C at position 931. Additionally, the term zygosity with respect to a GPR109A amino acid refers to homozygosity or heterozygosity of a particular amino acid at a position corresponding to a particular position of SEQ ID NO: 2. For instance, an isoleucine or a methionine at position 317, or a cysteine or an arginine at position 311 of SEQ ID NO:2.

The I317 polymorphism of the GPR109A gene can be characterized by a single nucleotide polymorphism whereby a guanine is substituted with an adenine at the nucleotide position corresponding to nucleotide position 951 of SEQ ID NO:1. This substitution results in replacement of the codon ATG with the codon ATA (i.e., a G to A transition), which results in substitution of methionine (“M”) in the wildtype sequence with an isoleucine (“I”) at amino acid position 317 (M317I). As described in detail in the Examples section below, in comparison with wildtype GPR109A, the I317 polymorphism does not change the ability of GPR109A to signal through Gi. However, in comparison with wildtype GPR109A, the I317 polymorphism blunts the ability of GPR109A to stimulate MAP kinase activity in response to niacin, leading to reduced niacin-mediated stimulation of MAP kinase activity (“decreased MAP kinase activity”). Therefore, and without being held to theory, an individual who is heterozygous or homozygous for the I317 GPR109A polymorphism can exhibit a diminished or reduced flushing effect in response to niacin or other niacin receptor agonist and is otherwise at reduced probability for an adverse reaction to the administration of a niacin receptor agonist. For example, an individual who is heterozygous or homozygous for the I317 GPR109A polymorphism can exhibit a reduced frequency of flushing (compared to the general population) in response to a niacin receptor agonist while still experiencing the favorable effects of niacin receptor-mediated signaling such as HDL elevation, atheroma regression and reverse cholesterol transport. Also, for example, an individual who is wild-type at position 317 can exhibit an increased frequency of flushing (compared to the general population) in response to a niacin receptor agonist while still experiencing the favorable effects of niacin receptor-mediated signaling such as HDL elevation, atheroma regression and reverse cholesterol transport.

The C311 polymorphism of the GPR109A gene can be characterized by a single nucleotide polymorphism whereby a C is substituted with a T at the nucleotide position corresponding to nucleotide position 931 of SEQ ID NO:1. This substitution results in replacement of the codon CGC with the codon TGC (i.e., a C to T transition), which results in substitution of a cysteine (“C”) for an arginine (“R”) in the wild type sequence at amino acid position 311 (R311C). As illustrated in the Examples section below, in comparison with wildtype GPR109A, the C311 polymorphism does not change the ability of GPR109A to signal through Gi. Notably, like the wildtype GPR109A, the C311 polymorphism does not change the ability of GPR109A to stimulate MAP kinase activity in response to niacin (“normal MAP kinase activity”). Therefore, and without being held to theory, an individual who is heterozygous or homozygous for the C311 GPR109A polymorphism is like an individual that is homozygous for the wildtype GPR109A gene, in that neither of these individuals exhibit a diminished flushing effect in response to niacin or other niacin receptor agonist, is not otherwise at reduced probability for an adverse reaction to the administration of a niacin receptor agonist, and can in fact be at an elevated probability, at least an elevated probability as in comparison to an individual that is homozygous or heterozygous for the I317 GPR109A polymorphism.

In view of this discovery, the invention provides assays, based on detection of a GPR109A polymorphism, for determining whether an individual in need of a treatment comprising administration of niacin or an analog thereof is at reduced probability for an adverse reaction to the niacin or said analog, e.g., determining the level of probability an individual has for experiencing flushing. More specifically, the present invention relates to assays for determining the presence or absence in a nucleic acid or protein sample of a polymorphism in GPR109A receptor. Detection of a GPR109A polymorphism can be accomplished using any of a variety of methods, which methods are well within the level of skill of the ordinary artisan in the relevant field. The present invention also relates to methods comprising the steps of obtaining GPR109A nucleic acid or amino acid sequence of an individual from a database and determining the presence or absence of a polymorphism in GPR109A receptor by inspection of said nucleic acid or amino acid sequence. Exemplary methods for detecting such GPR109A polymorphisms are described below.

The invention provides a method of correlating a polymorphism in a GPR109A receptor to a probability for a condition associated with stimulation of MAP kinase activity by niacin or a niacin analog. For example, the invention provides a method of correlating a polymorphism in a GPR109A receptor to a probability for cutaneous flushing induced by niacin or a niacin analog. The method includes, for example, introducing a polymorphism or polymorphisms into a GPR109A receptor sequence and determining the ability of the polymorphic GPR109A to activate the MAP kinase pathway induced by niacin or a niacin analog and comparing the level of MAP kinase activation to the level obtained using the reference GPR109A receptor of SEQ ID NO:2. A polymorphism in GPR109A that results in a blunted MAP kinase activation compared to the reference GPR109A receptor is correlated to a reduced probability for a condition associated with stimulation of MAP kinase activity by niacin or a niacin analog, such as cutaneous flushing.

The invention provides a method of determining a level of risk for an individual for a condition associated with stimulation of MAP kinase activity by niacin or a niacin analog, comprising obtaining a biological sample from the individual; identifying within said biological sample a nucleotide at a position corresponding to nucleotide position 951 of SEQ ID NO:1 or an amino acid at a position corresponding to amino acid position 317 of SEQ ID NO:2; and assigning the level of risk to the individual for the condition associated with stimulation of MAP kinase activity by niacin or the niacin analog.

In one embodiment, the nucleotide at the position corresponding to nucleotide position 951 of SEQ ID NO:1 or the amino acid at the position corresponding to amino acid position 317 of SEQ ID NO:2 is identified to be homozygous or heterozygous in the individual. In a further embodiment, an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced risk for the condition associated with stimulation of MAP kinase activity by niacin or the niacin analog.

In one embodiment, the homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced risk for the condition associated with stimulation of MAP kinase activity by niacin or the niacin analog. In another embodiment, the homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated risk for the condition associated with stimulation of MAP kinase activity by niacin or the niacin analog.

In another embodiment, a further step is added wherein a portion of the GPR109A gene spanning nucleotides 949 to 951 of SEQ ID NO:1 is amplified prior to the identifying step. In one embodiment, the portion of the GPR109A gene spanning nucleotides 949 to 951 of SEQ ID NO:1 is amplified by polymerase chain reaction (PCR). In another embodiment, the identifying is performed by a method selected from the group consisting of a hybridization assay, a sequencing assay, a microsequencing assay, a MALDI-TOF assay, and an allele-specific amplification assay. In a further embodiment, the identifying is performed by an antibody-based assay.

In one embodiment, the method is for use in predicting an individual's risk for the condition associated with the stimulation of MAP kinase activity by niacin or a niacin analog in a therapy for a lipid disorder, wherein said therapy comprises administration of an amount of niacin or the niacin analog. In another embodiment, the amount of niacin or the niacin analog is a therapeutically effective amount. In a further embodiment, the condition associated with stimulation of MAP kinase activity by niacin or the niacin analog is cutaneous flushing.

In addition, the invention provides the invention provides a method of determining a level of probability for an individual for a condition associated with stimulation of MAP kinase activity by niacin or a niacin analog, said method comprising the steps of: (a) obtaining a GPR109A receptor nucleic acid sequence or a GPR109A receptor amino acid sequence for the individual; (b) identifying within said GPR109A receptor nucleic acid sequence a nucleotide polymorphism compared to SEQ ID NO:1, or within said GPR109A receptor amino acid sequence an amino acid polymorphism compared to SEQ ID NO:2; and (c) assessing the ability of said GPR109A receptor nucleic acid sequence containing said nucleotide polymorphism or GPR109A receptor amino acid sequence containing said amino acid polymorphism to affect MAP kinase activation mediated by niacin, wherein a blunted MAP kinase activation compared to the MAP kinase activation of a GPR109A receptor containing SEQ ID NO:2 is associated with a decreased level of probability to the individual for the condition associated with stimulation of MAP kinase activity by niacin or the niacin analog. In one embodiment, the condition associated with the stimulation of MAP kinase activity by niacin or the niacin analog is cutaneous flushing.

### Nucleic Acid Assays

An aspect of the present invention is to provide assays for determining the presence or absence in a sample (e.g., a nucleic acid sample) of a polymorphism in a GPR109A gene. In one aspect, the assays of the invention are useful for determining the presence or absence of a codon encoding a polymorphic amino acid, e.g., the presence or absence of a codon encoding isoleucine at amino acid position 317 of GPR109A, where the codon encoding GPR109A I317 corresponds to nucleotides 949-951 of SEQ ID NO:1. The skilled artisan would be aware, for example, that isoleucine is encoded by each of the codons ATA, ATT and ATC. In one aspect, the assays of the present invention are useful for determining the presence or absence of a single nucleotide polymorphism (SNP) corresponding, for example, to GPR109A C311 or to GPR109A I317. In one aspect, the C311 polymorphism is characterized by substitution of a C with a T in wildtype GPR109A gene at the nucleotide position corresponding to nucleotide position 931 of SEQ ID NO:1. In one aspect, the I317 polymorphism is characterized by substitution of a G with an A, or a T, or a C in the wildtype GPR109A gene at the nucleotide position corresponding to nucleotide position 951 of SEQ ID NO:1. In one aspect, the I317 polymorphism is characterized by substitution of a guanine with an adenine in wildtype GPR109A gene at the nucleotide position corresponding to nucleotide position 951 of SEQ ID NO:1.

In accordance with one embodiment of the methods of the invention, a biological sample is obtained from an individual. Any biological sample that comprises a polynucleotide from the individual is suitable for use in the methods of the invention. The biological sample can be processed so as to isolate the polynucleotides therein. Alternatively, whole cells or other biological samples can be used without isolation of the polynucleotides contained therein. Methods for isolating genomic DNA and mRNA and for preparing cDNA from mRNA are well known in the art, and kits for carrying out such methods are commercially available.

In one embodiment, a sample of genomic DNA is obtained from an individual (e.g, an individual) and prepared in accordance with conventional methods, e.g., lysing cells, removing cellular debris, separating the DNA from proteins, lipids, or other components present in the mixture, optionally cleaving the isolated DNA, and assaying it against an oligonucleotide probe or probes of interest. Appropriate oligonucleotide probes can be isolated or manufactured according to methods well known in the art. See, for instance, Molecular Cloning, A Laboratory Manual, 2nd ed. (eds. Sambrook et al.) CSH Laboratory Press, Cold Spring Harbor, N.Y. 1989. Generally, at least about 0.1-5 μg of DNA will be employed, usually at least about 0.5 μg of DNA, while less than 50 μg of DNA will usually be sufficient.

The detection of a GPR109A polymorphism in the sample can be accomplished by any means well known in the art, including, but not limited to: determination of the nucleotide sequence of the polynucleotide sample; amplification of a sequence with specific primers; single strand conformational polymorphism analysis; hybridization analysis; denaturing gradient gel electrophoresis; mismatch cleavage detection; and the like.

For instance, the nucleic acid sequence for the region of interest can be amplified by conventional techniques. For example, a portion of the GPR109A gene spanning nucleotides 949 to 951 or 930 to 932 can be amplified prior to identifying the presence or absence of the nucleotide polymorphism of interest. The region of interest can be cloned into a suitable vector and grown in sufficient quantity. Alternatively, polymerase chain reaction (PCR) can be used to provide sufficient amounts for analysis. Once amplified, the sequence can then be determined using conventional methods, such as nucleic acid sequencing using a dideoxy chain termination method or other well-known methods. See, e.g., Example 4 infra.

Additionally, a variety of other automated sequencing procedures, known in the art, can be used to directly sequence the GPR109A gene, or a portion thereof in which a specific polymorphism is known to occur. Once sequenced, polymorphisms can be detected by comparing the sequence of the sample nucleic-acid with a reference nucleic acid sequence containing a GPR109A polymorphism. A C substituted with a T at a nucleotide position corresponding to nucleotide position 931 of SEQ ID NO:1 is indicative of a GPR109A C311 polymorphism. A G substituted with an adenine at a nucleotide position corresponding to nucleotide position 951 of SEQ ID NO:1 is indicative of a GPR109A I317 polymorphism.

Alternatively, once isolated a sample comprising nucleic acid can be amplified with primers that only amplify a region known to comprise a GPR109A polymorphism(s). Either genomic DNA or mRNA can be used directly. If the polymorphic region is not present amplification will not take place. In this regard, PCR can be used to determine whether a polymorphism is present by using a primer that is specific for the polymorphism. See, e.g., WO 94/16101; Cohen et al. (1996) Adv. Chromatography 36:127-162. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Such methods can comprise the steps of collecting from an individual a biological sample comprising the individual's genetic material as a template, optionally isolating template nucleic acid (genomic DNA, mRNA, or both) from the biological sample, contacting the template nucleic acid sample with one or more primers that specifically hybridize with a GPR109A polymorphic nucleic acid molecule under conditions such that hybridization and amplification of the template nucleic acid molecules in the sample occurs, and detecting the presence, absence, and/or relative amount of an amplification product and comparing the length to a control GPR109A sample.

Hence, observation of an amplification product of the expected size will be an indication that the GPR109A polymorphism contained within the GPR109A polymorphic primer is present in the test nucleic acid sample. Parameters such as hybridization conditions, GPR109A polymorphic primer length, and position of the polymorphism within the GPR109A polymorphic primer can be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230. For instance, in one specific embodiment, the primers should be constructed such that for the detection of a GPR109A C311 polymorphism the primer recognizes a T instead of a C in GPR109A gene at a nucleotide position corresponding to nucleotide position 931 of SEQ ID NO:1, wherein if a T is not present the extension terminates. For the detection of a GPR109A I317 polymorphism the primer should be designed to recognize an A, a T or a C instead of a G in GPR109A gene at a nucleotide position corresponding to nucleotide 951 of SEQ ID NO:1, wherein if an A, a T or a C respectively is not present the extension terminates. Thus, the length of the expression products will be indicative of whether the specified polymorphism is present or not. In one specific embodiment, for the detection of a GPR109A I317 polymorphism the primer should be designed to recognize an adenine instead of a guanine in GPR109A gene at a nucleotide position corresponding to nucleotide 951 of SEQ ID NO:1, wherein if an adenine is not present the extension terminates. Thus, the length of the expression products will be indicative of whether the specified polymorphism is present or not.

Alternatively, once the region comprising a suspected GPR109A polymorphism has been amplified, the presence or absence of a GPR109A polymorphism can be detected by SSCP analysis; denaturing gradient gel electrophoresis (DGGE); mismatch cleavage detection; and heteroduplex analysis in gel matrices. These techniques are well known in the art and a detailed description can be found in a variety of publications, including, e.g., “Laboratory Methods for the Detection of Mutations and Polymorphisms in DNA” (1997) G. R. Taylor, ed., CRC Press, and references cited therein; incorporated herein its entirety by reference. For instance, in performing SSCP analysis, the PCR product can be digested with a restriction endonuclease that recognizes a sequence within the PCR product generated by using as a template a reference GPR109A sequence, but does not recognize a corresponding PCR product generated by using as a template a variant GPR109A sequence (that is the C311C or I317 sequences) by virtue of the fact that the variant sequence does not contain a recognition site for the restriction endonuclease.

Additionally, a detectable label can be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label can be a two stage system, where the amplified DNA is conjugated to biotin, haptens, etc. having a high affinity binding partner, e.g. avidin, specific antibodies, etc., where the binding partner is conjugated to a detectable label. The label can be conjugated to one or both of the primers. Alternatively, the pool of nucleotides used in the amplification can be labeled, so as to incorporate the label into the amplification product.

In one embodiment, PCR is used in combination with matrix assisted laser desorption/ionization time-of-flight mass spectrometry (MALDI-TOF) to determine the presence of a GPR109A single nucleotide polymorphism. See, e.g., Tang et al., Journal of Proteome Research (2004) 3:218-227 and Storm et al., Methods Mol Biol (2003) 212:241-262. By way of illustration and not limitation, the MassARRAY Homogenous MassEXTEND™ Assay commercially available from Sequenom Inc. (San Diego, Calif.) can be used to determine, for example, the presence of an adenine in GPR109A gene at a nucleotide position corresponding to nucleotide position 951 of SEQ ID NO:1.

Hybridization with the variant sequence can also be used to determine the presence of a GPR109A polymorphism. Hybridization analysis can be carried out in a number of different ways; including, but not limited to: Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, can also be used as a means of detecting the presence of variant sequences. For instance, identification of a polymorphism in a nucleic acid sample can be performed by hybridizing both sample and control nucleic acids to high density arrays containing hundreds or thousands of oligonucleotide probes. Cronin et al. (1996) Human Mutation 7:244-255; and Kozal et al. (1996) Nature Med. 2:753-759. In one embodiment, the probes comprise short oligonucleotides of about 10 to about 50 nucleotides, or about 15 to about 30 nucleotides, or about 17 to about 19 nucleotides in length and typically span the variable position wherein the SNP can be located. In one embodiment, the probe can be configured such that the variable position is centrally disposed in the oligonucleotide; wherein the hybridization or lack of hybridization is informative as to the presence or absence of the particular polymorphism being reviewed.

Hybridization reactions can be performed under conditions of different stringency. Conditions that increase stringency of a hybridization reaction are widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. Examples of stringent conditions are hybridization and washing at 50° C. or higher and in 0.1×SSC (9 mM NaCl/0.9 mM sodium citrate).

A Tₘ is the temperature in degrees Celsius at which 50% of a polynucleotide duplex made of complementary strands hydrogen bonded in anti-parallel direction by Watson-Crick base pairing dissociates into single strands under conditions of the experiment. Tₘ can be predicted according to a standard formula, such as:

Tₘ=81.5+16.6 log [X⁺]+0.41 (% G/C)−0.61 (% F)−600/L
where [X⁺] is the cation concentration (usually sodium ion, Na⁺) in mol/L; (% G/C) is the number of G and C residues as a percentage of total residues in the duplex; (% F) is the percent formamide in solution (wt/vol); and L is the number of nucleotides in each strand of the duplex.

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al. Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

Microarrays

The methods of the invention can further include the use of an array of oligonucleotides (i.e., “probes”), where oligonucleotide probes at discrete positions on the array are complementary to one or more of the suspected wildtype or polymorphic sequences to be tested. Such an array can comprise a series of oligonucleotides, each of which can specifically hybridize to a different sequence, such as a different allelic variant or polymorphism. For examples of arrays, see Hacia et al. (1996) Nat. Genet. 14:441-447 and DeRisi et al. (1996) Nat. Genet. 14:457-460.

Thus, one or more polymorphic forms can be present in the array to serve as a probe. Accordingly, an array to be used in accordance with the methods of the invention can include nucleic acids encoding one or both GPR109A C311 and I317, as well as the wildtype GPR109A. In some embodiments, an array includes a combination of nucleic acids encoding at least 2 or 3 different GRP109A alleles. Arrays of interest can be addressable and can further comprise other genetic sequences of interest. The oligonucleotide probe sequence on the array should generally be at least about 5 to about 12 nt in length, at least about 15 nt, at least about 18 nt, at least about 20 nt, or at least about 25 nt, or can be the length of the provided polymorphic sequences, or can extend into the flanking regions to generate fragments of 100 to 200 nt in length. For examples of arrays, see Ramsay (1998) Nature Biotech. 16:40-44; Hacia et al. (1996) Nature Genetics 14:441-447; Lockhart et al. (1996) Nature Biotechnol. 14:1675-1680; and De Risi et al. (1996) Nature Genetics 14:457-460.

A number of methods are available for creating microarrays of biological samples and the probes to be used therewith, such as arrays of DNA samples to be used in DNA hybridization assays. Exemplary are PCT Application Serial No. WO95/35505, published Dec. 28, 1995; U.S. Pat. No. 5,445,934, issued Aug. 29, 1995; and Drmanac et al. (1993) Science 260:1649-1652. Yershov et al. (1996) Genetics 93:4913-4918 describe an alternative construction of an oligonucleotide array. The construction and use of oligonucleotide arrays is reviewed by Ramsay (1998) supra.

Methods of using high density oligonucleotide arrays are known in the art. For example, Milosavljevic et al. (1996) Genomics 37:77-86 describe DNA sequence recognition by hybridization to short oligomers. See also, Drmanac et al. (1998) Nature Biotech. 16:54-58; and Drmanac and Drmanac (1999) Methods Enzymol. 303:165-178; The use of arrays for identification of unknown mutations is proposed by Ginot (1997) Human Mutation 10:1-10.

Detection of known mutations is described in Hacia et al. (1996) Nat. Genet. 14:441-447; Cronin et al. (1996) Human Mut. 7:244-255; and others. The use of arrays in genetic mapping is discussed in Chee et al. (1996) Science 274:610-613; Sapolsky and Lishutz (1996) Genomics 33:445-456; etc. Quantitative monitoring of gene expression patterns with a complementary DNA microarray is described in Schena et al. (1995) Science 270:467. DeRisi et al. (1997) Science 270:680-686 explore gene expression on a genomic scale. Wodicka et al. (1997) Nat. Biotech. 15:1-15 perform genome wide expression monitoring in S. cerevisiae.

In one particular embodiment of the invention, a sample from an individual is obtained and processed according to techniques well known in the art so as to isolate the nucleic acid in the sample. The nucleic acids of the sample may or may not be cleaved. The nucleic acid samples are then denatured and labeled. Labeling can be performed according to methods well known in the art, using any method that provides for a detectable signal either directly or indirectly from the nucleic acid fragment. In a preferred embodiment, the fragments are end-labeled, in order to minimize the steric effects of the label. For example, terminal transferase can be used to conjugate a labeled nucleotide to the nucleic acid fragments.

Suitable labels include biotin and other binding moieties; fluorochromes, e.g. fluorescein isothiocyanate (FITC), rhodamine, Texas Red, phycoerythrin, allophycocyanin, 6-carboxyfluorescein (6-FAM), 2=,7=-dimethoxy-4=,5=-dichloro-6-carboxyfluorescein (JOE), 6-carboxy-X-rhodamine (ROX), 6-carboxy-2=,4=,7=,4,7-hexachlorofluorescein (HEX), 5-carboxyfluorescein (5-FAM) or N,N,N=,N=-tetramethyl-6-carboxyrhodamine (TAMRA), and the like. Where the label is a binding moiety, the detectable label is conjugated to a second stage reagent, e.g. avidin, streptavidin, etc. that specifically binds to the binding moiety, for example a fluorescent probe attached to streptavidin. Incorporation of a fluorescent label using enzymes such as reverse transcriptase or DNA polymerase, prior to any fragmentation of the sample, is also possible.

Oligonucleotide probes encoding the sequence to be tested (e.g., coding for GPR109A wildtype, C311, or I317 cDNA or mRNA) are fabricated into an array on a substrate. The labeled genomic sample is washed across the array of oligonucleotide probes and hybridization is allowed to occur. Hybridization of the labeled sequences is accomplished according to methods well known in the art. As set forth above, hybridization can be carried out under conditions varying in stringency, preferably under conditions of high stringency, e.g. 6×SSPE, at 65° C., to allow for hybridization of any complementary sequences having extensive homology, usually having no more than one or two mismatches in a probe of 25 nucleotides in length, i.e. at least 95% to 100% sequence identity. It is understood that the above is for purpose of illustrations.

Such high density microarrays of oligonucleotides are well known in the art and are commercially available. Alternatively, methods of producing large arrays of oligonucleotides are described in U.S. Pat. No. 5,134,854 (Pirrung et al.), and U.S. Pat. No. 5,445,934 (Fodor et al.) using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in International Patent application WO 95/35505.

The sequence of oligonucleotides on the array will correspond to the known target sequences of one or more of the allelic variants of interest, i.e., nucleic acid sequences encoding the polymorphic or wildtype regions. The length of oligonucleotide present on the array is an important factor in how sensitive hybridization will be to the presence of a mismatch. Usually oligonucleotides will be at least about 12 nucleotides (nts) in length, more usually at least about 15 nt in length, preferably at least about 20 nt in length and more preferably at least about 25 nt in length, and will be not longer than about 35 nt in length, usually not more than about 30 nt in length.

Microarrays can be scanned to detect hybridization of the labeled genomic samples. Methods and devices for detecting fluorescently marked targets on devices are known in the art. Generally such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al. (1996) Genome Res. 6:639. A scan, using the appropriate excitation line, is performed for each fluorophore used. The digital images generated from the scan are then combined for subsequent analysis. For any particular array element, the ratio of the fluorescent signal from one Nucleic acid sample is compared to the fluorescent signal from the other Nucleic acid sample, and the relative signal intensity determined.

Methods for analyzing the data collected by fluorescence detection are known in the art. Data analysis includes the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

### Polypeptide-Based Assays

The invention also explicitly contemplates detecting a polymorphic GPR109A receptor polypeptide and/or a GPR109A amino acid polymorphism as a means of predicting an individual's probability of having an adverse reaction, e.g. flushing, to a niacin receptor agonist. As discussed in detail supra, the polymorphic GPR109A receptors of the invention differ by at least one polymorphic amino acid. Accordingly, it is possible to generate antibodies that specifically bind a polymorphic GPR109A receptor polypeptide so as to distinguish a GPR109A amino acid polymorphism (e.g., I317) from the corresponding wildtype amino acid residue (e.g., M317) or from other GPR109A amino acid polymorphisms (e.g., C311).

GPR109A receptor polypeptides can be used to produce antibodies according to methods well known in the art. As used herein, the term “antibodies” includes antibodies of any isotype, fragments of antibodies which retain specific binding to antigen, including, but not limited to, Fab, Fv, scFv, and Fd fragments, fusion proteins comprising such antibody fragments, detectably labeled antibodies, and chimeric antibodies and unless explicitly stated otherwise encompasses polyclonal antibodies and monoclonal antibodies. “Antibody specificity”, in the context of antibody-antigen interactions, is a term well understood in the art, and indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies for a polymorphic GPR109A receptor polypeptide and/or for a particular GPR109A amino acid polymorphism (e.g., I317). Antibodies of the invention can originate from any suitable animal including, but not limited to, rabbit, mouse, rat, and hamster.

Antibodies are prepared in accordance with conventional methods well known in the art. For preparing antibodies, the polymorphic or wildtype GPR109A receptor polypeptide itself or a peptide which comprises the GPR109A receptor polypeptide and spans a GPR109A amino acid polymorphism (e.g., I317) or the corresponding wildtype amino acid residue (e.g., M317) is used as immunogen directly or conjugated to a known immunogenic carrier, e.g., KLH, pre-S HBsAg, other viral or eukaryotic proteins, or the like. In particular embodiment, various adjuvants can be employed, with a series of injections, as appropriate. In the case of monoclonal antibodies, after one or more booster injections, the spleen is isolated, the lymphocytes immortalized by cell fusion, and then screened for high affinity antibody binding. The immortalized cells, i.e., hybridomas, producing the desired antibodies can then be expanded. If desired, the mRNA encoding the monoclonal antibody heavy and light chains can be isolated and mutagenized by cloning in E. coli, and the heavy and light chains mixed to further enhance the affinity of the antibody. For further description, see Antibodies: A Laboratory Manual, Harlow and Lane eds., Cold Spring Harbor Laboratories, Cold Spring Harbor, N.Y., 1988.

Antibodies can be attached, directly or indirectly (e.g., via a linker molecule) to a solid support for use in an assay to determine and/or measure the presence of a polymorphic GPR109A receptor polypeptide in a biological sample and/or to determine and/or measure the presence of a particular GPR109A amino acid polymorphism (e.g., I317) or the corresponding wildtype amino acid residue (e.g., M317) in a biological sample. Attachment is generally covalent, although it need not be. Solid supports include, but are not limited to, beads (e.g., polystyrene beads, magnetic beads, and the like); plastic surfaces (e.g., polystyrene or polycarbonate multi-well plates typically used in an ELISA or radioimmunoassay (RIA), and the like); sheets, e.g., nylon, nitrocellulose, and the like; and chips, e.g., SiO₂ chips such as those used in microarrays. Accordingly, the invention further provides assay devices comprising antibodies specific for a polymorphic GPR109A receptor polypeptide attached to a solid support and/or for a particular GPR109A amino acid polymorphism (e.g., I317) or the corresponding wildtype amino acid residue (e.g., M317).

A single antibody or a battery of different antibodies can then be used to create an assay device. Such an assay device can be prepared using conventional technology known to those skilled in the art. The antibody can be purified and isolated using known techniques and bound to a support surface using known procedures. The resulting surface having antibody bound thereon can be used to assay a test sample, e.g., a biological sample, in vitro to determine if the sample contains one or more types of GPR109A polymorphic polypeptides. For example, antibodies which bind only to a specific GPR109A polymorphic epitope can be attached to the surface of a material. Alternatively, a plurality of specific antibodies, which can be arranged in an array, wherein antibodies specific for two or more different GPR109A polymorphic epitopes are attached to the solid support, can be used. A test sample is brought into contact with the antibodies bound to the surface of material. Specific binding can be detected using any known method. If specific binding is not detected, it can be deduced that the sample does not contain the specific GPR109A polymorphic epitope. As one non-limiting example of how specific binding can be detected, once the test sample has been contacted with the antibodies bound to the solid support, a second, detectably-labeled antibody can be added, which recognizes a GPR109A epitope distinct from the epitope recognized by the solid support-bound antibody.

A variety of other reagents can be included in the assays to detect GPR109A polymorphic polypeptides described herein. These include reagents such as salts, neutral proteins, e.g. albumin, detergents, etc., that are used to facilitate optimal protein-protein binding, and/or reduce non-specific or background interactions. Reagents that improve the efficiency of the assay, such as protease inhibitors, anti-microbial agents, etc. can be used. The components are added in any order that provides for the requisite binding. Incubations are performed at any suitable temperature, typically between 4° C. and 40° C. Incubation periods are selected for optimum activity, but can also be optimized to facilitate rapid high-throughput screening. Typically between 0.1 and 1 hour will be sufficient.

Selection of Therapy Based on Niacin Receptor Genotype

The methods of the invention can also be applied to facilitate the selection of a therapy for conditions in which stimulation of a niacin receptor can be beneficial. Niacin favorably affects various lipid-associated disorders; including lipoprotein metabolism. However, providing information to the clinician regarding the propensity of an individual to experience adverse side effects, such as flushing, is of value in selecting a therapy regimen from which the individual will benefit most and/or with which the patient will most likely comply with a prescribed regimen. Thus, it is of interest to provide a method for assessing the level and propensity of flushing in selecting a niacin-receptor based therapy or prescribing an alternate therapy.

Stimulation of the niacin receptor has been shown to be beneficial in treatment of various lipid-associated disorders. As used herein the term “lipid-associated disorder” means any disorder related to a non-optimal level of an atherosclerosis associated serum lipid, for example, LDL-cholesterol, VLDL-cholesterol, HDL-colesterol, Lp(a), or triglycerides in an individual or a condition associated with abnormally high levels of circulating lipids. Therefore, a lipid-associated disorder can be, for example, dyslipidemia with an elevated level of LDL-cholesterol, a reduced level of HDL-cholesterol, or disorders that are caused, at least in part, by a non-optimal level of an atherosclerosis associated serum lipid such as atherosclerosis, metabolic syndrome, heart attack (myocardial infarction), or stroke. Non-optimal levels of these lipids or less than optimal ratios of these lipids are considered to be lipid-associated disorders.

Dyslipidemia is a general term for abnormal concentrations of serum lipids such as HDL (low), LDL (high), VLDL (high), triglycerides (high), lipoprotein (a) (high), free fatty acids (high) and other serum lipids, or combinations thereof. For example, an individual with dyslipidemia can have a high level of total cholesterol compared with the optimum level (hypercholesterolemia). Atherosclerosis refers to a form of vascular disease characterized by the deposition of atheromatous plaques containing cholesterol and lipids on the innermost layer of the walls of large and medium-sized arteries. Metabolic syndrome is characterized by a group of metabolic risk factors including: central obesity, atherogenic dyslipidemia, raised blood pressure, insulin resistance or glucose intolerance, prothrombotic state, and proinflammatory state. Other disorders associated with sub-optimal levels of serum lipid include coronary artery disease (CAD) or coronary heart disease, congestive heart failure, angina, aneurysm, ischemic heart disease, myocardial infarction and stroke.

As discussed above, therapeutic doses of niacin or other niacin receptor agonists alter serum lipid levels and function to alleviate the symptoms associated with the various lipid-associated disorders. However, stimulation of the niacin receptor, particularly at the large doses of niacin required, is frequently associated with adverse side effects. Side effects can include gastrointestinal disturbances, liver toxicity, and disruption of glucose metabolism and uric acid levels. The most frequent and prominent side effect of niacin therapy is intense flushing, often accompanied by cutaneous itching, tingling and warmth. Although the flushing reaction is generally harmless, it is sufficiently unpleasant that patient compliance is markedly reduced. Often, 30-40% of patients cease taking niacin treatment within days after initiating therapy. Accordingly, a therapeutically effective amount is an amount that is sufficient for obtaining a desired response or effect, for instance, and not to be limited hereby, an amount sufficient to lower serum lipid levels and to alleviate the symptoms associated with the various lipid-associated diseases, or an amount sufficient to effectively reduce a flushing response.

Identifying which individuals are at risk and the level of probability or risk of flushing or other adverse side effects provides the clinician the opportunity to counsel the individual prior to or during niacin receptor agonist therapy. Further, the clinician can prescribe additional drug or non-drug based therapies to mitigate such adverse effects. Thus, identifying the level of probability for individuals for an adverse side effect can facilitate improved individual compliance with a niacin receptor agonist therapy, and can identify those individuals who would most likely be in need of therapy to mitigate side effects. In addition, identifying which individuals are at risk of niacin-induced flushing or other adverse side effects provides the clinician the opportunity to prescribe non-niacin based therapies, for example, treatment with drugs that target statins.

Thus, an additional aspect of the invention is directed to methods for treating an individual having, or suspected of having, a lipid associated disorder. The methods generally comprise analyzing a biological sample from an individual who is a candidate for a niacin receptor agonist therapy (e.g., niacin or niacin analog therapy), where generally such candidates are suspected of having, or have been clinically diagnosed with, a lipid-associated disorder. The biological sample is analyzed for the presence or absence of a GPR109A I317 gene polymorphism. The presence of a GPR109A I317 gene polymorphism indicates that the subject has a lower level of probability of (or susceptibility to) an adverse side effect caused by stimulation of a GPR109A receptor, and thus, for example, higher doses of a niacin receptor agonist can be administered with less risk of the subject experiencing an adverse flushing reaction. The absence of a GPR109A I317 gene polymorphism indicates that the subject has an elevated probability of (or has an elevated susceptibility to) an adverse side effect caused by stimulation of a GPR109A receptor, and thus therapies based on, for example, high doses niacin or administration of a niacin receptor agonist that stimulates a level of MAP kinase activity associated with flushing can be contraindicated or can fail due to, for example, lack of compliance by the individual due to an adverse flushing reaction following administration. A treatment plan that is most effective for individuals clinically diagnosed as having a lipid-associated disorder is then selected on the basis of the detected GPR109A polymorphism.

Additionally, in another aspect, the invention provides methods for tailoring an individual's prophylactic or therapeutic treatment with niacin receptor modulators according to that individual's GPR109A genotype. A niacin receptor modulator is material, for example a ligand or compound, which modulates or changes an intracellular response when it binds to a niacin receptor. An intracellular response can be, for example, a change in GTP binding to membranes or modulation of the level of a second messenger such as cAMP.

Pharmacogenomics allows a clinician or physician to target prophylactic or therapeutic treatments to individuals who will most benefit from the treatment and to avoid treatment of individuals who will experience symptomatic side effects, in the case of niacin receptor agonist the adverse side effect can be cutaneous flushing. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a niacin receptor modulator as well as tailoring the dosage, regimen, and/or therapeutically effective amounts to be administered so as to attain the effect desired by treatment with the modulator (e.g., a reduction in the amount of circulating lipids, or a reduced flushing response, etc.).

A determination of how a given GPR109A polymorphism is predictive of an individual's likelihood of responding to a given drug treatment for a condition relating to abnormally high levels of circulating lipids can be accomplished by determining the genotype of the individual in the GPR109A gene, as described above, and/or determining the effect of the drug on MAP kinase enzymatic activity. Information generated from one or more of these approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating an individual with a niacin receptor modulator, such as niacin or an analog thereof.

Accordingly, in one embodiment, the method involves determining the presence or absence of a GPR109A gene polymorphism in the individual and selecting a treatment plan for the individual, based on the GPR109A genotype, so as to avoid the risk of the adverse side effects; where the particular polymorphism indicates a risk or a non risk of an adverse side effect following administration of an GPR109A agonist. It is expressly contemplated that in said method involving determining the presence of a GPR109A gene polymorphism, said determining can comprise the steps of obtaining a GPR109A nucleic acid or amino acid sequence of an individual from a database and determining the presence or absence of GPR109A gene polymorphism by inspection of said nucleic acid or amino acid sequence in said database, where the particular polymorphism indicates a risk or a non risk of an adverse side effect following administration of a GPR109A agonist. For instance, it is contemplated that the nucleic acid and/or amino acid sequences of an individual can be stored in a computer readable media that is searchable. Hence, in one embodiment, a method for determining a polymorphism in a GPR109A gene can involve searching the database to identify a GPR109A genetic sequence that can then be scanned to determine the presence or the absence of a GPR109A polymorphism, wherein a complete nucleic acid of a GPR109A gene, or a partial sequence of a GPR109A gene, that overlaps with the region containing the polymorphism, can be searched in the database. Likewise, in another embodiment, a method for determining a polymorphism in the GPR109A amino acid sequence can involve searching the database to identify a GPR109A amino acid sequence that then can be scanned to determine the presence or the absence of a GPR109A polymorphism, wherein a complete GPR109A or a partial GPR109A amino acid sequence that overlaps with the region containing the polymorphism can be searched in the database.

### Clinical Trial Design

The invention further provides for methods of grouping or “segmenting” subjects, according to the subject's GPR109A genotype or phenotype and/or their level of probability for experiencing an adverse side effect, for example on the basis of suitability or unsuitability for a clinical trial. The GPR109A genotype of each subject in a pool of potential subjects for a clinical trial can be classified. Subjects that are homozygous or heterozygous for the wildtype GPR109A receptor or homozygous or heterozygous for a GPR109A polymorphism (e.g., I317) can be classified accordingly so as to provide an individual population that is more homogenous for a GPR109A genotype and phenotype.

For example, subjects who are heterozygous or homozygous for the GPR109A I317 polymorphism can be identified and separated from those subjects that do not carry the GPR109A I317 polymorphism. Those subjects that do not carry the GPR109A I317 polymorphism can then be selected for participation in an investigative or clinical trial of a niacin receptor agonist having reduced adverse side effects (e.g., flushing). Subjects that do not carry the GPR109A I317 polymorphism should provide a population exhibiting MAP kinase activity-associated side effects (e.g., flushing) in a more predictable manner. By excluding subjects having the GPR109A I317 polymorphism, the efficacy of the niacin receptor agonist for treating a disorder, e.g. a lipid disorder, with no or with reduced adverse side effects, e.g. flushing, can be more readily and accurately assessed. In one aspect, the lipid disorder is selected from elevated levels of circulating or non-circulating low density lipoprotein (LDL) cholesterol, elevated levels of circulating or non-circulating free fatty acids, elevated levels of circulating or non-circulating triglycerides, low levels of circulating or non-circulating high density lipoprotein (HDL) cholesterol, abnormally high levels of circulating or non-circulating lipids (e.g., arteriosclerosis).

The GPR109A genotype classification of an individual can also be used in assessing the efficacy of a niacin receptor agonist in a heterogeneous subject population. Thus, comparison of an individual's GPR109A genotype relative to that of others in the subject population facilitates analysis of results and provides better support for analysis of the niacin receptor agonist-based therapeutic regimens that are efficacious for a particular subject or subject population.

In addition, GPR109A genotype classification of an individual can also be used to identify a population for assessment of candidate therapies for mitigation of adverse side effects, e.g. flushing, that result from niacin receptor agonist therapy. For example, subjects who are heterozygous or homozygous for the GPR109A I317 polymorphism can be identified and separated from those subjects that do not carry the GPR109A 317 polymorphism. Those subjects that do not carry the GPR109A I317 polymorphism can then be selected for inclusion in a clinical trial to assess the efficacy of a therapy (e.g., a drug based therapy) for reducing adverse side effects that result for niacin receptor activation. Subjects that do not carry the GPR109A I317 polymorphism should provide a population exhibiting MAP kinase activity-associated side effects (e.g., flushing) in a more predictable manner. By excluding subjects having the GPR109A I317 polymorphism, the effects of the candidate therapies to mitigate side effects (e.g., flushing) of a niacin receptor agonist therapy can be more readily and accurately assessed. In one aspect, the candidate therapy is an antagonist of prostaglandin D2 activity. In another aspect, the candidate therapy is a partial agonist of the GPR109A receptor.

In addition, GPR109A genotype classification of an individual can also be used to identify a population for assessment of candidate therapies for treating schizophrenia. For example, an initial subject population exhibiting an impaired flushing response to niacin can be identified, independently of GPR109A genotype. From this initial subject population are then removed those subjects that are either homozygous or heterozygous for a GPR109A polymorphism (e.g., I317) associated with a decreased MAP kinase-associated flushing response to niacin. The remaining subjects represent a subset of the initial subject population identified for inclusion in a clinical trial to assess the efficacy of a therapy (e.g., a drug based therapy) for treating schizophrenia. By excluding subjects having a decreased flushing response to niacin attributable to a GPR109A polymorphism (e.g., I317), the effects of the candidate therapies to treat schizophrenia can be more readily and accurately assessed. In related aspect, it is expressly contemplated that by so excluding subjects having a decreased flushing response to niacin attributable to a GPR109A polymorphism (e.g., I317), the value of an impaired skin flush response to niacin as a diagnostic for schizophrenia (see, e.g., WO 97/45145 and Puri B K et al., Int J Clin Pract (2001) 55:368-370) is increased. In one aspect, the schizophrenia is acute first-episode schizophrenia.

Additionally, subjects who are homozygous for the GPR109A I317 polymorphism can be identified for inclusion in a clinical trial, where those subjects who are not homozygous for the GPR109A I317 polymorphism are separated and excluded. Those subjects who are homozygous for the GPR109A I317 polymorphism can then be selected for participation in an investigative or clinical trial wherein it is of interest to have subjects that are at a lower probability of exhibiting the flushing reaction. For instance, a clinical trial such as a therapy involving niacin or an analog thereof, including combination therapies, wherein the issue of flushing based non-compliance is sought to be avoided by only including those subjects that are not at risk of flushing (i.e., those that are homozygous for the GPR109A I317 polymorphism). By excluding subjects who are not homozygous for the GPR109A I317 polymorphism, the efficacy of a drug or a combination of drugs, for instance niacin or an analog thereof, for treating a disorder, e.g. a lipid disorder, can be tested with a lower risk of flushing based non-compliance.

In addition, the ability to target individuals expected to show the most clinical benefit, based on their GPR109A genotype can enable: 1) the repositioning of already marketed drugs; 2) the rescue of drug candidates whose clinical development has been discontinued as a result of efficacy limitations (e.g., due to poor compliance), which are patient subgroup-specific; and 3) an accelerated and less costly development for candidate therapeutics and more optimal drug labeling (e.g. since measuring the effect of various doses of an agent on patients with a particular expression profile is useful for optimizing effective dose).

Also provided are methods for the post hoc analysis of data, such as clinical data from a clinical trial, wherein results from a clinical trial involving an individual(s) that is untyped as to the subject's GPR109A genotype, or not classified as to the probability of flushing with respect to that genotype, are obtained and then the clinical results are analyzed with respect to (e.g., associated or linked with) the presence or absence of a GPR109A polymorphism in the subject. For instance, a trial participant, trial clinician or both can be blinded, or uninformed, as to the genotype of the participant. Once the results of the trial are obtained an analysis of those results can then take into account data related to the participant's GPR109A genotype.

Accordingly, in one representative embodiment, the subject invention is directed to determining a level of probability for a condition associated with stimulation of MAP kinase activity by niacin or a niacin analog for an individual having a GPR109A receptor zygosity, which includes identifying a clinical outcome for each of a plurality of patients in a clinical trial involving a therapy, wherein the therapy includes the administration of an amount of niacin or a niacin analog, and wherein the clinical outcome is exhibiting or not exhibiting the condition associated with the stimulation of MAP kinase activity by niacin or the niacin analog. Once the therapy results are obtained the GPR109A receptor zygosity for each of the plurality of patients in the clinical trial is identified, and the clinical outcome is associated with the GRP109A receptor zygosity for each of said plurality of patients.

The associated clinical outcomes and GPR109A receptor zygosities can then be analyzed so as to allow assignment of a level of probability for the condition associated with stimulation of MAP kinase activity by niacin or the niacin analog for the individual having the GPR109A receptor zygosity. This analysis can involve segmenting or not segmenting the clinical outcomes on the basis of the GPR109A receptor zygosity so as to thereby make a segmented group and an unsegmented group; and comparing the clinical outcomes for the segmented group with the clinical outcomes for the unsegmented group.

In one embodiment, the invention provides a kit for use in the methods of the invention, for example, a kit for determining a level of probability for an individual for a condition associated with stimulation of MAP kinase activity by niacin or a niacin analog, a kit for using a GPR109A receptor zygosity of an individual for determining a suitability or an unsuitability of an individual for inclusion in a clinical trial, or a kit for determining a level of probability for a condition associated with stimulation of MAP kinase activity by niacin or a niacin analog for an individual having a GPR109A receptor zygosity. A kit can comprise reagents and instructions for performing the methods of the invention. For example, a kit can include genotyping reagents such as reagents for isolating nucleic acid molecules and reagents for amplifying nucleic acid molecules such as primers. A kit can also include, for example, a MAP kinase assay such as an ELISA. In addition, a kit can contain control samples, for example, to show that amplification reactions are not contaminated.

The contents of the kit are contained in packaging material, preferably to provide a sterile, contaminant-free environment. In addition, the packaging material contains instructions indicating how the materials within the kit can be employed. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

In one embodiment, the invention provides a method of determining a level of probability for an individual for flushing induced by niacin or a niacin analog, said method comprising the steps of: (a) obtaining a GPR109A receptor nucleic acid sequence or a GPR109A receptor amino acid sequence for the individual; (b) identifying within said nucleic acid sequence or said amino acid sequence a nucleotide at a position corresponding to nucleotide position 951 of SEQ ID NO:1 or an amino acid at a position corresponding to amino acid position 317 of SEQ ID NO:2; and (c) assigning the level of probability to the individual for flushing induced by niacin or the niacin analog, wherein said assigning is based on correlation of said nucleic acid sequence or said amino acid sequence with a niacin-induced signal. In one embodiment, said niacin-induced signal is MAP kinase activation. In another embodiment, said niacin-induced signal is a calcium flux. In a further embodiment, said niacin-induced signal is enhanced cAMP signaling via a Gs pathway.

In one embodiment, the nucleotide at the position corresponding to nucleotide position 951 of SEQ ID NO:1 or the amino acid at the position corresponding to amino acid position 317 of SEQ ID NO:2 is identified to be homozygous or heterozygous in the individual. In another embodiment, an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for flushing induced by niacin or the niacin analog. In a further embodiment, homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for flushing induced by niacin or the niacin analog. In another embodiment, homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for flushing induced by niacin or the niacin analog.

In one embodiment, said method is for use in predicting an individual's probability for flushing induced by niacin or the niacin analog in a therapy for a lipid disorder, wherein said therapy comprises administration of an amount of niacin or the niacin analog. In one embodiment, the amount of niacin or the niacin analog is a therapeutically effective amount. In another embodiment, said GPR109A receptor nucleic acid sequence or said GPR109A amino acid sequence is obtained from a database. In a further embodiment, said method is for use in selection of a therapy comprising administration of an amount of niacin or a niacin analog for a lipid disorder, wherein said therapy is selected so as to ameliorate flushing induced by niacin or the niacin analog.

In one embodiment, said method is for use in determining a suitability or an unsuitability of the individual for inclusion in a clinical trial for assessing an efficacy of an amount of a GPR109A receptor agonist for treating or preventing a lipid disorder without or with less flushing induced by niacin or the niacin analog. In another embodiment a zygosity of the individual is indicative of the individual being unsuitable for inclusion in the clinical trial, said zygosity being selected from the group consisting of: (a) homozygosity for an adenine at a nucleotide position corresponding to nucleotide position 951 of SEQ ID NO:1 or for an isoleucine at an amino acid position corresponding to amino acid position 317 of SEQ ID NO:2; and (b) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2.

In one embodiment wherein a zygosity of the individual is indicative of the individual being suitable for inclusion in the clinical trial, said zygosity being homozygosity for a guanine at a nucleotide position corresponding to nucleotide position 951 of SEQ ID NO:1 or for a methionine at an amino acid position corresponding to amino acid position 317 of SEQ ID NO:2. In another embodiment, said method is for use in determining a suitability or an unsuitability of the individual for inclusion in a clinical trial for assessing an efficacy of a compound for ameliorating flushing induced by niacin or the niacin analog. In one embodiment, the compound is an inhibitor of D2 activity. In one embodiment, a zygosity of the individual is indicative of the individual being unsuitable for inclusion in the clinical trial, said zygosity being selected from the group consisting of: (a) homozygosity for an adenine at a nucleotide position corresponding to nucleotide position 951 of SEQ ID NO:1 or for an isoleucine at an amino acid position corresponding to amino acid position 317 of SEQ ID NO:2; and (b) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2.

In another embodiment, a zygosity of the individual is indicative of the individual being suitable for inclusion in the clinical trial, said zygosity being homozygosity for a guanine at a nucleotide position corresponding to nucleotide position 951 of SEQ ID NO:1 or for a methionine at an amino acid position corresponding to amino acid position 317 of SEQ ID NO:2. In one embodiment, said method is for use in determining a suitability or an unsuitability of the individual for inclusion in a clinical trial for assessing the efficacy of a compound for treating or preventing schizophrenia, wherein the individual exhibits a decreased flushing response to the administration of niacin or a niacin analog. In another embodiment, a zygosity of the individual is indicative of the individual being unsuitable for inclusion in the clinical trial, said zygosity being selected from the group consisting of: (a) homozygosity for an adenine at a nucleotide position corresponding to nucleotide position 951 of SEQ ID NO:1 or for an isoleucine at an amino acid position corresponding to amino acid position 317 of SEQ ID NO:2; and (b) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2. In one embodiment, a zygosity of the individual is indicative of the individual being suitable for inclusion in the clinical trial, said zygosity being homozygosity for a guanine at a nucleotide position corresponding to nucleotide position 951 of SEQ ID NO:1 or for a methionine at an amino acid position corresponding to amino acid position 317 of SEQ ID NO:2. In another embodiment, said method is for use in classifying the individual according to a level of probability for flushing induced by niacin or the niacin analog.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Methods and Materials

The following methods and materials are used as indicated in the Examples below.

Antibody Based Assays to Determine Induction of MAP Kinase

MAP kinase activity of wild type GPR109A and polymorphic GPR109A can be evaluated by ELISA and Western Blot assays as detailed below. In the examples shown below, an ELISA was used.

MAP Kinase ELISA:

A kit from Biosource (phosphoERK1/2 pT185pY187 ELISA, Catalog #K10-0091) was used according to the protocol set out below. The cells were serum-starved overnight prior to stimulating the cells with compound.

Compound Preparation and Cell Treatment:

Compounds were dissolved in DMSO. Do not go over 1% DMSO because higher DMSO concentrations will stress the cells and activate MAPK. PMA (100 ng/ml) was used as a positive control.

Cell dishes were taken out of the incubator and placed on a rocker set to gentle rocking. Compound was carefully added and cells were returned to the incubator to incubate for 5 min. At 4.5 min, the medium was aspirated from dishes in the order that the compound was added. Then 2 ml cold PBS was added and excess medium was removed by gentle swirling. The PBS was aspirated and 1 ml of PBS was added (1 ml for confluent 6 cm dish).

Cell Extraction Buffer

Cell extraction buffer was prepared as follows:

| | |
|---|---|
| 10 mM Tris pH 7.4 | 5 ml (1 M) |
| 100 mM NaCl | 10 ml (5 M) |
| 1 mM EDTA pH 8.0 | 1 ml (0.5 M) |
| 20 mM Na4P2O7 | 100 ml (100 mM) |
| 1% TX-100 | 5 ml (100%) |
| 10% glycerol | 50 ml (100%) |
| 0.1% SDS | 5 ml (10%) |
| 0.5% Deoxycholate | 2.5 g |
| | 500 ml final volume |

Add fresh:

| | |
|---|---|
| 2 mM Na3VO4 | 1 ml (100 mM) |
| 1 mM PMSF | 250 ul (200 mM) |
| 25 ug/ml Leupeptin | 125 ul (10 ug/ul) |
| 25 ug/ml Aprotinin | 125 ul (10 ug/ml) |
| | 50 ml final volume |

Cell Collection and Extraction: on Ice

Cells were scraped from dish with a rubber policeman and transferred to a microfuge tube, then centrifuged at 3000 rpm at 4° C. for 5 minutes. The PBS was aspirated and cell pellet lysed in Cell Extraction Buffer (0.1% SDS) (250-300 μl for confluent 6 cm dish) for 30 min. on ice with vortexing at 10 min intervals.

The mixture was then centrifuged at max speed (16,000×G) for 15 min. at 4° C. Clarified lysates were transferred to new microfuge tubes and protein concentration measure. To measure protein, samples were diluted with Cell Extraction Buffer to a concentration of 1 mg/ml then boiled for 5 min. After cooling, they were centrifuged at max speed for 5 min at room temperature. Lysates were diluted 1:10 with Standard Diluent Buffer to a concentration of 0.1 mg/ml (0.01% SDS final) and loaded 100 μl in duplicate to sample wells (10 μg/well). Lysates can be stored at −80° C.

Reagent Preparation and Storage:

Reconstitution and dilution of phospho ERK1/2 standard: Phospho ERK1/2 standard was reconstituted with 1.2 ml Standard Diluent Buffer, mixed gently and allowed to sit for 10 min. to ensure complete reconstitution. This stock is 100 U/ml.

In duplicate, 125 μl of Standard Diluent Buffer was added to wells B-H of master plate (not ELISA plate). 250 μl of 100 U/ml stock was added to well A. Serial dilutions

(1:2) were made by transferring 125 μl of 100 U/ml in well A to well B, mixing and transferring 125 μl of well B to well C and so on until well G. Well H was not diluted (0 U/ml).

Storage and final dilution of rabbit IgG HRP: α-Rabbit IgG HRP concentrate was brought to room temperature and gently mixed. Then Mix 10 μl of concentrate was mixed with 1 ml of HRP Diluent for each 8-well strip used in assay.

Dilution of wash buffer: The 25× wash buffer concentrate was brought to RT and mixed to ensure full reconstitution. Wash buffer concentrate was diluted with deionized water (40 ml 25×/960 ml H₂O).

Assay Method: Procedure and Calculations:

Standard and sample application: All reagents were at room temperature and mixed before use. Microtiter plates were at room temperature before opening foil bags. The number of 8 well strips needed for assay was determined and bag was sealed and returned to 4° C. 100 μl of standard (prepared in 3A2) was added in duplicate (28-well strips). Two wells were left empty for chromogen blank. 100 μl of samples were added in duplicate to sample wells. The plate was covered with plate cover and tapped gently on side of plate to mix. Plates were incubated at room temperature for 2 hours. (The plate can be incubated overnight at 4° C.).

Washes: Liquid from wells was aspirated with aspirator. The wells were filled with 200 μl of diluted wash buffer. After incubation for 30 sec. the liquid was aspirated. This was repeated 4 times.

Detection antibody: 100 μl of αphosphoERK1/2 solution was pipetted into each well except the chromogen blanks. The cover was replaced and tapped gently to mix. Incubation occurred for 1 hour at room temperature.

Washes: The wells were washed 4 times as above.

αRabbit IgG HRP: 100 μl of αRabbit IgG HRP working solution was added to each well except the chromogen blanks. The cover was replaced and tapped gently to mix. Incubation occurred for 30 min at room temperature.

Washes: The wells were washed 4 times as above.

Chromogen. 100 μl of Stabilized Chromogen was added to each well. Incubation occurred for 20 min. at room temperature in dark (foil or metal was not used).

Stop solution: 100 μl of Stop Solution was added to each well and tapped to mix the plate. Reading the plate. The plate was read at an absorbance of 450 nm.

MAP Kinase Western Blot:

Reagents: The following reagents are used in the MAP kinase Western blot.

MAP kinase Western Blot Reagents

1% NP-40 Lysis buffer:
1% NP-40
20 mM Tris pH 8.0
100 mM NaCl
1 mM EDTA
1 mM PMSF
200 μM Na₃V0₄
10 U/ml Aprotinin
10 μg/ml Leupeptin
SDS-PAGE Running buffer:
14.4 g Glycine
3.03 g Tris base
1 g or 5 ml (20%) SDS
q.s. to 1 liter with water
1 X Transfer buffer:
100 ml 10X Transfer buffer
200 ml MeOH
700 ml water
TBS/Tween:
100 ml 10 X TBS
900 ml water
500 ul Tween20 (100%)
5X Laemmli Sample buffer:
300 Mm Tris pH 6.8
25% Glycerol
10% SDS
0.05% Bromophenol Blue
120 ul/ml final volume of stock 2-βME
10 X Transfer buffer:
0.2 M Tris base
1.92 M Glycine
10 X TBS:
60.5 g Tris base
87.5 g NaCl
q.s to 1 Liter with water
BLOCKO:
4% BSA in TBS/Tween

Sample preparation: Sample preparation is performed on ice. Medium is aspirated off cells and cells rinsed with PBS. Cells are harvested in appropriate volume of 1% NP-40 lysis buffer (volume depends on dish size, cell density, etc). Typically, 500 μl is used for a confluent 6 cm dish. Lysate is transferred into a microfuge tube. The tube is vortexed and incubated on ice for 30 min. and centrifuged at max speed, 4° C. for 10 min.

Protein assay: Stock protein standard BSA is prepared @ 1.41 μg/μl in water. 14.2 μl stock standard is added to 485.8 μl water=40 μg/ml. 200 μl of 40 μg/ml standard is added to well 9A and 9B. 100 μl of water is added to 1-8, rows A and B. A serial dilution is performed by adding 100 μl of 40 μg/ml to the 20 μg/ml well, mixing and transferring 100 μL into next well until you reach the 0.31 μg/ml well. The last 100 μl from the 0.31 ug/ml well is discarded. 99 μl of water is added to the wells designated for unknowns. 1 μl of unknown sample is added to wells in triplicate. 25 μl of 5× Bradford dye reagent is added to standards and unknowns. Incubation occurs at room temperature for at least 5 minutes. Absorbance is read at 595λ.

Sample dilution and preparation for loading: Samples are diluted to a final concentration of 1 μg/μl with water or lysis buffer. 5× Laemmli sample buffer is added, and samples are vortexed and boiled for 5 min.

Set up for NOVEX gels: The white adhesive strip at the bottom of gel is pulled off. The comb is gently pulled out. Gels are rinsed with water and placed in gel box. The inner reservoir is filled with Running buffer and the outer reservoir filled above the gel opening (where the white strip was). The wells are flushed with a syringe.

Loading samples: Samples are loaded being careful not to spill over into the adjacent wells. Standard markers are loaded and empty wells loaded with 1× sample buffer.

Running the gel: The gels are run at 150V for 1.5 hr.

Transfer to Nitrocellulose (0.2 μm pore size): 1× transfer buffer is prepared and the gel, sponges, Whatman paper and nitrocellulose membranes are soaked in transfer buffer. Layering is done in the following order: positive electrode, sponges, membrane, gel, sponges, negative electrode. Outer and inner chamber is filled with transfer buffer. The transfer occurs at 25 V for 1.5 hour.

Blocking of membrane: The transfer rig is dismantled. Nitrocellulose membrane is placed in BLOCKO and incubated overnight at 4° C. on a rocker.

Primary antibody: The membrane is washed 1×10 min. with TBS/tween on a rocker. Primary antibody is diluted in BLOCKO™. Incubation occurred on a rocker for 2 hr at room temperature.

Secondary antibody: The membrane is washed 2×15 min with TBS/tween on a rocker. Secondary antibody is diluted in TBS/tween. Incubation occurred on a rocker for 1 hr at room temperature.

Detection: The membrane is washed 3×15 min with TBS/tween on a rocker and then rinsed once with water. Chemiluminescent detection reagent is added (10 ml ECL reagent+5 ul H₂O₂ (30%) per membrane) and rocked for 2 min. The membrane is placed in plastic sheet protector and excess detection reagent and bubbles are squeezed out. The membrane is then exposed to film.

### Example 1

### Preparation of GPR109A R311C and GPR109A M317I

Several naturally occurring polymorphisms in GPR109A have been identified. In order to examine the effects of such polymorphisms upon the GPR109A receptor, nucleotide substitutions were introduced into the coding region for wild type GPR109A to generate various polymorphisms for testing, including the polymorphism C311 and I317.

Nucleotide substitutions were made using QuikChange™ Site-Directed™ Mutagenesis Kit (Stratagene) according to manufacturer's instructions. The coding region for wild type GPR109A was used as template, and two mutagenesis primers were utilized, as well as a selection marker oligonucleotide (included in kit). For convenience, the codon mutation incorporated into GPR109A C311 or GPR109A I317 and the respective oligonucleotides are noted, in standard form, in the table below:

| | | 5′-3′ orientation | 5′-3′ orientation | Cycle Conditions |
|---|---|---|---|---|
| | | (sense), (antisense), | Min (′), Sec (″) | |
| Receptor | Codon | (SEQ ID NO), | (SEQ ID NO), | cycles 2-4 |
| Identifier | Mutation | mutation bolded | mutation bolded | repeated 16 times |
| GPR109A | R311C | CTCCACTTTGATC | TCCTCTGGAGGCA | 98° for 2′ |
| C311 | | AACTGCTGCCTCC | GCAGTTGATCAA | 98° for 30″ |
| | | AGAGGA | AGTGGAG | 58° C. for 30″ |
| | | (SEQ ID NO:3) | (SEQ ID NO:4) | 72° for 12′ |
| | | | | 72° for 10′ |
| GPR109A | M317I | CCTCCAGAGGAAG | ATCTGGCTCACCT | 98° for 2′ |
| I317 | | ATAACAGGTGAGC | GTTATCTTCCTCT | 98° for 30″ |
| | | CAGAT | GGAGG | 58° C. for 30″ |
| | | (SEQ ID NO:7) | (SEQ ID NO:8) | 72° for 12′ |
| | | | | 72° for 10′ |

GPR109A C311 and GPR109A I317 encoding-polynucleotides produced by mutagenesis were then sequenced. The nucleic acid and deduced amino acid sequences for GPR109A C311 and GPR109A I317 were confirmed and are listed in the accompanying “Sequence Listing” appendix to this patent document. The nucleic acid sequence for GPR109A C311 is SEQ ID NO:5 and the amino acid sequence for GPR109A C311 is SEQ ID NO:6. The nucleic acid sequence for GPR109A I317 is SEQ ID NO:9 and the amino acid sequence for GPR109A I317 is SEQ ID NO:10.

### Example 2

### Effect of M317I GPR109A Amino Acid Polymorphism on Niacin-Mediated MAP Kinase Activation

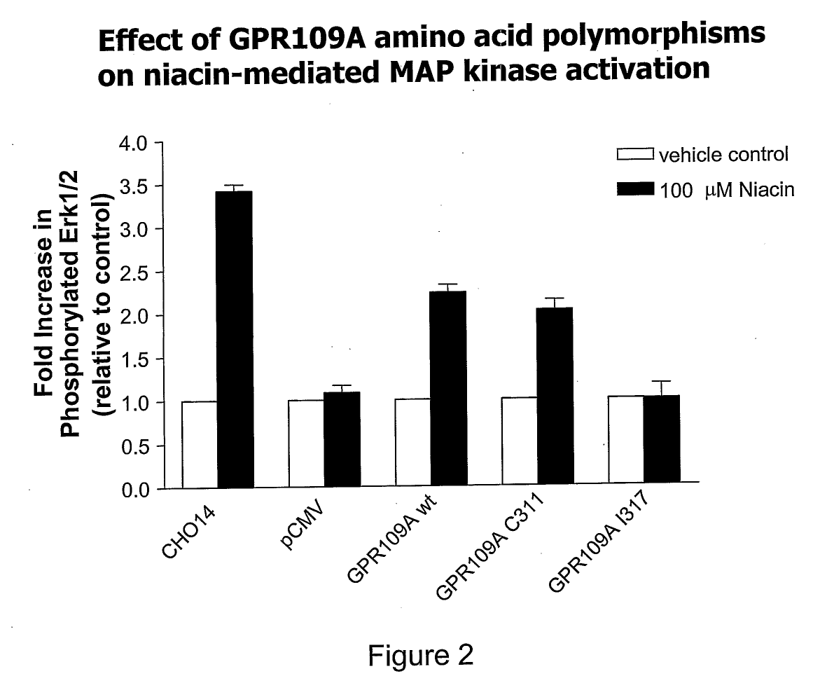

Human HEK293 cells were transfected with either pCMV vector or a cDNA plasmid selected from the group consisting of wild type GPR109A (“GPR109A wt”), R311C GPR109A (“GPR109A C311”), and M317I GPR109A (“GPR109A I317”). Transfection was carried out using Lipofectamine (Invitrogen). Forty-eight hours after transfection, the cells were stimulated with vehicle or with 100 μM niacin and MAP kinase activity determined by ELISA as described above. CHO cells stably transfected with wild type GPR109A (“CHO14”) were included in the assay as a positive control Results are presented in FIG. 2.

As shown in FIG. 2, the C311 polymorphism does not affect MAP kinase activation mediated by niacin. In contrast, the I317 polymorphism has a blunted response to niacin in the MAP kinase signaling pathway.

### Example 3

### Effect of M317I GPR109A Amino Acid Polymorphism on Niacin-Mediated Decrease in Intracellular Camp

Thyroid-stimulating hormone (TSH, or thyrotropin) receptor (TSHR) causes the accumulation of intracellular cAMP on activation by its ligand TSH. An effective technique for measuring the decrease in production of cAMP corresponding to activation of a Gi-coupled receptor such as GPR109A is to co-transfect TSHR with the Gi-coupled receptor and to carry out the assay in the presence of TSH to raise the level of basal cAMP, whereby TSHR acts as a “signal window enhancer.” Such an approach was used here.

Human HEK293 cells were co-transfected with thyroid-stimulating hormone

(TSH, or thyrotropin) receptor (TSHR) and either pCMV vector or a cDNA plasmid selected from the group consisting of wild type GPR109A, R311C GPR109A, and M317I GPR109A. Transfection was carried out using Lipofectamine (Invitrogen). Forty-eight hours after transfection, the cells were stimulated with various concentrations of niacin and 100 nM TSH (Sigma) for 1 h before whole cell cAMP was determined using the Adenylyl Cyclase Flashplate Assay kit from Perkin Elmer catalog #:SMP004B], as described below.

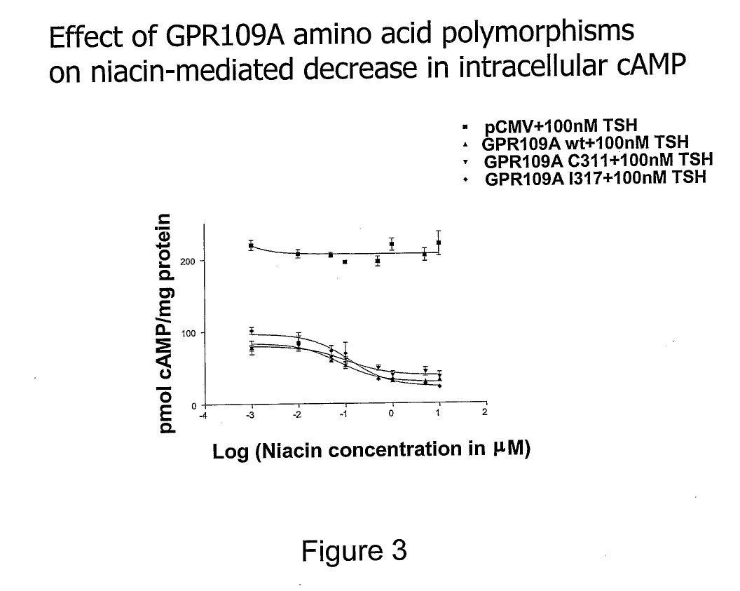

The transfected cells were placed into anti-cAMP antibody-coated wells that contained 100 nM TSH and either niacin at various concentrations or vehicle. All conditions were tested in triplicate. After a 1 h incubation at room temperature to allow for stimulation of cAMP, a Detection Mix (provided in the Perkin Elmer kit) containing ¹²⁵I-cAMP was added to each well and the plate was allowed to incubate for another hour at room temperature. The wells were then aspirated to remove unbound ¹²⁵I-cAMP. Bound ¹²⁵I-cAMP was detected using a Wallac Microbeta Counter. The amount of cAMP in each sample was determined by comparison to a standard curve, obtained by placing known concentrations of cAMP in some wells on the plate. Results are presented in FIG. 3.

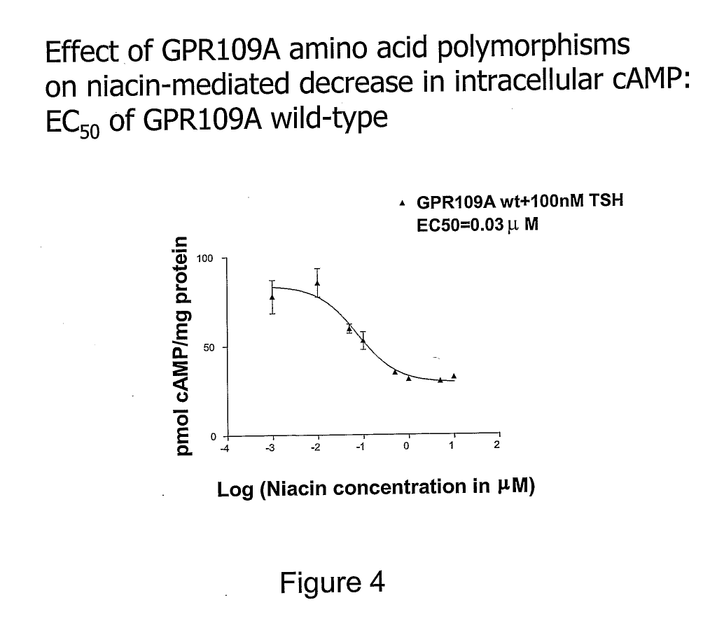

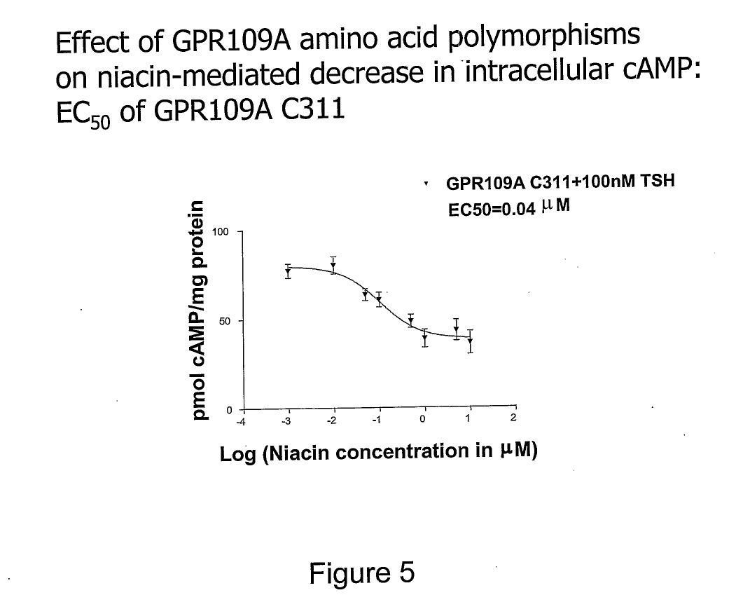

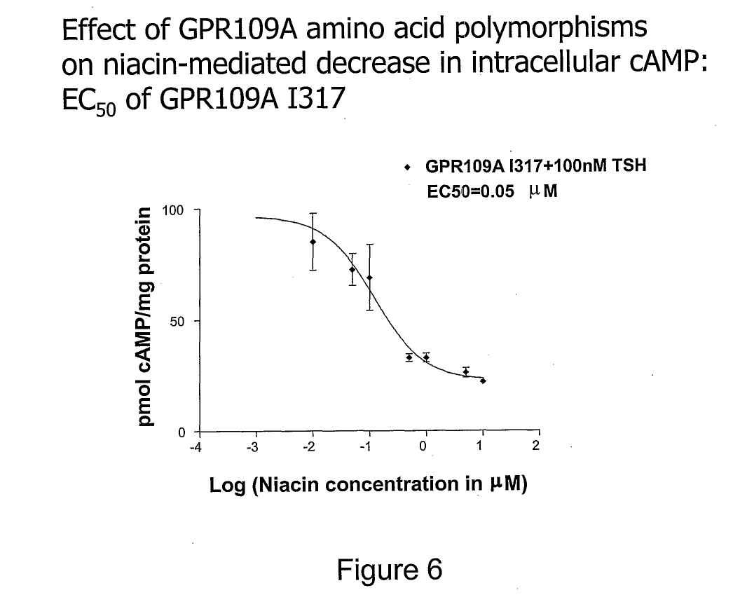

As shown in FIG. 2, the C311 polymorphism does not affect MAP kinase activation mediated by niacin while the I317 polymorphism has a blunted response to niacin in the MAP kinase signaling pathway. On the other hand both C311 and I317 polymorphic forms are capable of signaling through Gi with comparable EC50 values to wild type R311/M317 version as shown in FIGS. 3, 4, 5, and 6.

Thus the C311 polymorphic form has similar niacin-mediated signaling properties as the wild type GPR109A. While the I317 polymorphic form has diminished MAP kinase signaling yet maintains the Gi signaling capability in response to niacin. Because flushing is associated with the level of MAP kinase signaling, individuals who carry a I317 polymorphic form of the GPR109A are at reduced probability of flushing, while maintaining responsiveness to agonists of the receptor.

### Example 4

### Haplotype and Zygosity Frequencies of M317 and I317 for GPR109A

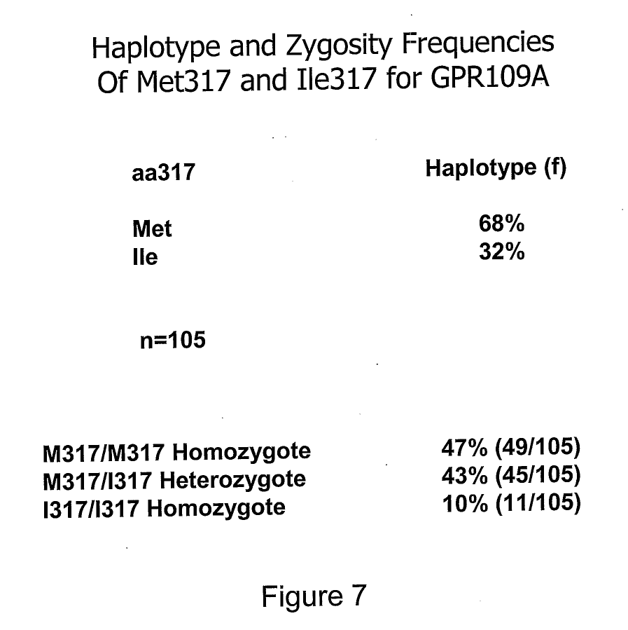

Genomic DNA was isolated from either blood samples of volunteers (38 total blood samples) or cadaver tissue samples (67 total tissue samples) using the FlexiGene DNA kit from Qiagen (Cat. # 51206). A specific region of GPR109A gene containing the M/I317 amino acid coding sequence was amplified by PCR using the Platinum PCR Supermix (Invitrogen Cat. # 11306-016) and the following primer set. Sense primer has the sequence 5′-GATGCCGATCCAGAATGGCGG-3′ (SEQ ID NO:11) and the antisense primer has the sequence 5′-TTCTTGGCATGGTTATTTAAGGAG-3′ (SEQ ID NO:12). The cycling condition was 25 cycles of 95° C. for 40 sec, 60° C. for 50 sec and 72° C. for 40 sec after an initial denaturation step at 94° C. for 4 min. The 610 bp amplified fragment was cloned using TOPO Cloning Kits (Invitrogen Cat # K4575-J10). Eight to ten independent clones from each genomic sample were sequenced to estimate what percent of the general population has Ile versus Met at amino acid position 317 (i.e., I317 versus M317) in GPR109A receptor. With eight independent clones being sequenced from each individual the probability of missing one of the two alleles is less than 0.4% (or 1 in 28). The results are presented in FIG. 7.

In the population sample studied in FIG. 7, M317 and I317 haplotype frequencies were found to be about 68% and about 32% respectively. About 47% of the individuals studied were found to be homozygous for M317, about 10% of the individuals studied were found to be homozygous for I317, and about 43% of the individuals studied were found to be heterozygous for M317 and I317. By way of comparison, in the study by Zellner et al. [Hum Mutat (2005) 25:18-21], M317 and I317 haplotype frequencies were found to be about 50% and about 42% respectively in their population sample.

### Example 5

### Correlation of GPR109A Genotype with Monocyte/Macrophage Phenotype

In this example, blood is drawn from individuals and used to: (1) determine the GPR109A genotype of the individual, as described above, and (2) isolate monocytes and macrophages. The isolated monocytes and macrophages are then assayed for MAPK activity, calcium flux, and enhanced cAMP signaling via a Gs pathway (for example, using isoproterenol to stimulate the Gs pathway via β2 adrenergic receptors). The genotype of the individual is then correlated with the phenotype of monocytes and macrophages from the individual. It is expected that the M317 polymorphism will correlate with a functional niacin-mediated SIGNAL response (for example, MAPK activation, calcium flux, and cAMP superstimulation) and the I317 polymorphism will correlate with a non-functional niacin-mediated SIGNAL response. The effect of heterozygosity will be determined. For example, heterozygotes can have a fully functional niacin-mediated SIGNAL response or a niacin-mediated SIGNAL response that is intermediate between a M317 homozygote and I317 homozygote.

Monocyte/Macrophage Isolation

Density gradient centrifugation. Peripheral blood mononuclear cells (PBMC's) are isolated from whole blood collected from healthy volunteers by density gradient centrifugation. Briefly, red blood cells are obtained by sedimentation of whole blood with 6% dextran for 1 hr. The resulting leukocyte-rich top layer is centrifuged for 10 min at 1000 rpm, washed with PBS, re-spun, and then resuspended in 12 mL PBS. The cell suspension is then gently overlayed on a 6 mL Ficoll solution and centrifuged for 30 min at 2000 rpm. The upper layer is then aspirated, leaving the interphase containing the PBMC's intact. The PBMC layer is then transferred to a conical tube with Hanks Balanced Salt Solution, bringing the final volume to 30 mL. Cells are counted, spun at 1000 rpm for 10 min, and resuspended in a PBS/2% BSA/2 mM EDTA solution at a concentration of 1×10⁸ cells per 300 μL.

Monocyte isolation. Monocytes are negatively selected through indirect labeling and magnetic separation of non-monocytes using the Human Monocyte Isolation Kit II (Miltenyi Biotec Inc.) according to manufacturer's instructions. Briefly, 100 μL of FcR blocking reagent and biotin antibody cocktail (including antibodies against CD3, CD7, CD16, CD19, CD56, CD123, and glycophorin A) are incubated with 1×10⁸ cells, obtained through density gradient centrifugation, for 10 min at 4° C. Cells are then incubated with 300 μL of PBS/2% BSA/2 mM EDTA solution and 200 μL of anti-biotin microbeads for 10 min at 4° C. Cell volume is adjusted to 30 mLs by adding PBS/2% BSA/2 mM EDTA solution, followed by centrifugation at 300×g for 10 min. The supernatant is discarded and cells are resuspended in 500 μL of PBS/2% BSA/2 mM EDTA solution. Up to 1 mL of cell solution is then loaded onto an LS column and allowed to completely elute before washing with PBS/2% BSA/2 mM EDTA. Cells collected in effluent are then plated in RPMI supplemented with 10% heat-inactivated bovine calf serum, 1% penicillin-streptomycin, and 1 mM sodium pyruvate. To induce macrophage differentiation, 10 ng/mL human GM-CSF is added to culture medium.

MAP Kinase Assays.

MAP kinase assays are performed using the phospho MAP kinase ELISA assay kit from Biosource (Cat# KHO 0091) according to the manufacturer's protocol. Specifically, either floating monocytes or adherent macrophages are treated overnight with 100 ng/mL of interferon-gamma, then serum-starved for 3-5 hrs immediately before performing the assay. Cells are stimulated with compounds for 5 min at 37° C. In case of adherent cells the medium is aspirated and the cells are rinsed with PBS. The cells are scraped in 1 ml PBS and transferred to a microfuge tube. In case of floating monocytes cells are harvested by centrifugation (2000 rpm, 5 min), washed with cold PBS and processed similar to the adherent cells. The cells are centrifuged for 5 min at 3000 rpm and the pellet is resuspended in 200 μl cell extraction buffer (0.1% SDS). The samples are incubated on ice for 30 min then clarified by centrifugation for 10 min, 4° C. at 13,000 rpm. Protein concentration is determined by Bradford analysis and 10 μg of protein is added to wells (96 well plate) coated with phospho MAP kinase capture antibody. The samples are incubated for 2 hr at RT then extensively washed before incubation with the phospho MAP kinase detection antibody for 1 hr at RT. The samples are washed then incubated with a rabbit HRP-conjugated secondary antibody for 30 min at RT. The samples are washed then incubated with chromogen in the dark for 20 min at RT before stopping the reaction with stop buffer. The plate is read at an absorbance of 450 nm.

Measurements of Ca²⁺ Flux by FLIPR

Ca²⁺ fluxes are measured via FLIPR equipment (Molecular Devices) using the Calcium 3 Assay Kit (Molecular Devices). For adherent cells, cells are plated on 384 black, clear-bottom plates and incubated for different periods of time in the presence of 10 ng/ml GM-CSF. Cells are stimulated the day before the assay with 100 ng/ml interferon γ (Ifnγ). On the day of the assay, cells are washed with BSA buffer (1× Hanks Buffered Saline Solution (HBSS)), 20 mM HEPES, 0.4 mg/ml BSA, 2.5 mM probenecid, pH 7.4). Then 25 μL of BSA buffer and 25 μL of Calcium 3 dye in HBSS buffer (1×HBSS, 20 mM HEPES, pH 7.4) is added to the cells and cells are incubated for 90 min at 37° C. Subsequently, 25 μL of compound in BSA buffer is added to cells and the FLIPR signal is read and analyzed. For floating monocytic cells, cells are washed with BSA buffer and diluted to the appropriate density in BSA buffer. After combining cells and Calcium 3 dye (25 μL each) cells are spun to the bottom of the plate by centrifugation (900 rpm, 5 min). Labeling and stimulation is performed as for the adherent cells.

Measurement of Cyclic AMP

cAMP assays are performed using the HTRF cAMP2 Dynamic Kit (CysBio) according to the manufacturer recommended protocol. Specifically, macrophage are stimulated overnight with 100 ng/ml interferon γ. On the day of the assay cells are collected by centrifugation, washed and resuspended in PBS. Cells (20K/well, 96 well plate) are combined with 2 μM Prostaglandin E2, indicated concentration of niacin and cAMP-D2 conjugate. After incubation for 10 min at RT, equal volume of Eu3+-cryptate labeled anti-cAMP antibody solution is added and incubated for 1 hr at RT. HTRF signal is read on a Pherastar Reader (BMG Labtech) and cAMP levels are calculated based on a cAMP standard curve.

While the present invention has been described with reference to the specific embodiments thereof, it should be understood by those skilled in the art that various changes can be made and equivalents can be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

What is claimed is:

1. A method of determining an individual's probability for a condition associated with a functional niacin receptor-mediated signal response, said method comprising the steps of:
(a) obtaining a GPR109A receptor nucleic acid sequence or a GPR109A receptor amino acid sequence for the individual;
(b) identifying within said nucleic acid sequence or said amino acid sequence a nucleotide at a position corresponding to nucleotide position 951 of SEQ ID NO:1 or an amino acid at a position corresponding to amino acid position 317 of SEQ ID NO:2; and
(c) assigning the level of probability to the individual for the condition associated with a functional niacin receptor-mediated signal response.

2. The method of claim 1, wherein the nucleotide at the position corresponding to nucleotide position 951 of SEQ ID NO:1 or the amino acid at the position corresponding to amino acid position 317 of SEQ ID NO:2 is identified to be homozygous or heterozygous in the individual.

3. The method of claim 1, wherein an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with a functional niacin receptor-mediated signal response.

4. The method of claim 1, wherein homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with a functional niacin receptor-mediated signal response.

5. The method of claim 1, wherein homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with a functional niacin receptor-mediated signal response.

6. The method of claim 1, wherein said method is for use in predicting an individual's probability for the condition associated with a functional niacin receptor-mediated signal response in a therapy for a lipid disorder, wherein said therapy comprises administration of an amount of niacin or the niacin analog.

7. The method of claim 1, wherein said GPR109A receptor nucleic acid sequence or said GPR109A amino acid sequence is obtained from a database.

8. The method of claim 1, wherein the condition associated with a functional niacin receptor-mediated signal response is HDL elevation.

9. The method of claim 1, wherein the condition associated with a functional niacin receptor-mediated signal response is atheroma regression.

10. The method of claim 1, wherein the condition associated with a functional niacin receptor-mediated signal response is reverse cholesterol transport.

11. A method of determining a level of probability for an individual for a condition associated with stimulation of MAP kinase activity by niacin or a niacin analog, said method comprising the steps of:
(a) obtaining a biological sample from the individual;
(b) identifying within said biological sample a nucleotide at a position corresponding to nucleotide position 951 of SEQ ID NO:1 or an amino acid at a position corresponding to amino acid position 317 of SEQ ID NO:2; and
(c) assigning the level of probability to the individual for the condition associated with stimulation of MAP kinase activity by niacin or the niacin analog.

12. The method of claim 11, wherein the nucleotide at the position corresponding to nucleotide position 951 of SEQ ID NO:1 or the amino acid at the position corresponding to amino acid position 317 of SEQ ID NO:2 is identified to be homozygous or heterozygous in the individual.

13. The method of claim 11, wherein an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with stimulation of MAP kinase activity by niacin or the niacin analog.

14. The method of claim 12, wherein homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with stimulation of MAP kinase activity by niacin or the niacin analog.

15. The method of claim 12, wherein homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with stimulation of MAP kinase activity by niacin or the niacin analog

16. The method of claim 11, further comprising a step wherein a portion of the GPR109A gene spanning nucleotides 949 to 951 of SEQ ID NO:1 is amplified prior to said identifying step.

17. The method of claim 16, wherein said portion of the GPR109A gene spanning nucleotides 949 to 951 of SEQ ID NO:1 is amplified by polymerase chain reaction (PCR).

18. The method of claim 11, wherein said identifying is performed by a method selected from the group consisting of a hybridization assay, a sequencing assay, a microsequencing assay, a MALDI-TOF assay, and an allele-specific amplification assay.

19. The method of claim 11, wherein said identifying is performed by an antibody-based assay.

20. The method of claim 11, wherein said method is for use in predicting an individual's probability for the condition associated with stimulation of MAP kinase activity by niacin or a niacin analog in a therapy for a lipid disorder, wherein said therapy comprises administration of an amount of niacin or the niacin analog.

21. A method of determining a level of probability for an individual for a condition associated with stimulation of MAP kinase activity by niacin or a niacin analog, said method comprising the steps of:
(a) obtaining a GPR109A receptor nucleic acid sequence or a GPR109A receptor amino acid sequence for the individual;
(b) identifying within said nucleic acid sequence or said amino acid sequence a nucleotide at a position corresponding to nucleotide position 951 of SEQ ID NO:1 or an amino acid at a position corresponding to amino acid position 317 of SEQ ID NO:2; and
(c) assigning the level of probability to the individual for the condition associated with stimulation of MAP kinase activity by niacin or the niacin analog.

22. The method of claim 21, wherein the nucleotide at the position corresponding to nucleotide position 951 of SEQ ID NO:1 or the amino acid at the position corresponding to amino acid position 317 of SEQ ID NO:2 is identified to be homozygous or heterozygous in the individual.

23. The method of claim 21, wherein an adenine at the nucleotide position corresponding to nucleotide position 951 of SEQ ID NO:1 or an isoleucine at the amino acid position corresponding to amino acid position 317 of SEQ ID NO:2 is indicative of the individual being at reduced probability for the condition associated with stimulation of MAP kinase activity by niacin or the niacin analog.

24. The method of claim 22, wherein homozygosity or heterozygosity of an adenine at the nucleotide position corresponding to nucleotide position 951 of SEQ ID NO:1 or of an isoleucine at the amino acid position corresponding to amino acid position 317 of SEQ ID NO:2 is indicative of the individual being at reduced probability for the condition associated with stimulation of MAP kinase activity by niacin or the niacin analog.

25. The method of claim 22, wherein homozygosity of a guanine at the nucleotide position corresponding to nucleotide position 951 of SEQ ID NO:1 or of a methionine at the amino acid position corresponding to amino acid position 317 of SEQ ID NO:2 is indicative of the individual being at elevated probability for the condition associated with stimulation of MAP kinase activity by niacin or the niacin analog

26. The method of claim 21, wherein said method is for use in predicting an individual's probability for the condition associated with stimulation of MAP kinase activity by niacin or a niacin analog in a therapy for a lipid disorder, wherein said therapy comprises administration of an amount of niacin or the niacin analog.

27. The method of claim 21, wherein said GPR109A receptor nucleic acid sequence or said GPR109A amino acid sequence is obtained from a database.

28. The method according to claim 1, 11, or 21, wherein said method is for use in selection of a therapy comprising administration of an amount of niacin or a niacin analog for a lipid disorder, wherein said therapy is selected so as to ameliorate a condition associated with stimulation of MAP kinase activity by niacin or the niacin analog.

29. The method according to claim 1, 11, or 21, wherein said method is for use in determining a suitability or an unsuitability of the individual for inclusion in a clinical trial for assessing an efficacy of an amount of a GPR109A receptor agonist for treating or preventing a lipid disorder without or with less of a condition associated with stimulation of MAP kinase activity by niacin or a niacin analog.

30. The method according to claim 29, wherein a zygosity of the individual is indicative of the individual being unsuitable for inclusion in the clinical trial, said zygosity being selected from the group consisting of:
(a) homozygosity for an adenine at a nucleotide position corresponding to nucleotide position 951 of SEQ ID NO:1 or for an isoleucine at an amino acid position corresponding to amino acid position 317 of SEQ ID NO:2; and
(b) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2.

31. The method according to claim 29, wherein a zygosity of the individual is indicative of the individual being suitable for inclusion in the clinical trial, said zygosity being homozygosity for a guanine at a nucleotide position corresponding to nucleotide position 951 of SEQ ID NO:1 or for a methionine at an amino acid position corresponding to amino acid position 317 of SEQ ID NO:2.

32. The method according to claim 1, 11, or 21, wherein said method is for use in determining a suitability or an unsuitability of the individual for inclusion in a clinical trial for assessing an efficacy of a compound for ameliorating a condition associated with stimulation of MAP kinase activity by niacin or the niacin analog.

33. The method according to claim 32, wherein the compound is an inhibitor of prostaglandin D2 activity.

34. The method according to claim 32, wherein a zygosity of the individual is indicative of the individual being unsuitable for inclusion in the clinical trial, said zygosity being selected from the group consisting of:
(a) homozygosity for an adenine at a nucleotide position corresponding to nucleotide position 951 of SEQ ID NO:1 or for an isoleucine at an amino acid position corresponding to amino acid position 317 of SEQ ID NO:2; and
(b) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2.

35. The method according to claim 32, wherein a zygosity of the individual is indicative of the individual being suitable for inclusion in the clinical trial, said zygosity being homozygosity for a guanine at a nucleotide position corresponding to nucleotide position 951 of SEQ ID NO:1 or for a methionine at an amino acid position corresponding to amino acid position 317 of SEQ ID NO:2.

36. The method according to claim 1, 11 or 21, wherein said method is for use in classifying the individual according to a level of probability for a condition associated with stimulation of MAP kinase activity by niacin or a niacin analog.

37. A method of using a GPR109A receptor zygosity of an individual for determining a suitability or an unsuitability of the individual for inclusion in a clinical trial, wherein said zygosity is indicative of a level of probability for the individual for a condition associated with the stimulation of MAP kinase activity by niacin or a niacin analog.

38. The method of claim 37, wherein said GPR109A receptor zygosity is selected from the group consisting of:
(a) homozygosity for an adenine at a nucleotide position corresponding to nucleotide position 951 of SEQ ID NO:1 or for an isoleucine at an amino acid position corresponding to amino acid position 317 of SEQ ID NO:2;
(b) heterozygosity for an adenine at a nucleotide position corresponding to nucleotide position 951 of SEQ ID NO:1 or for an isoleucine at an amino acid position corresponding to amino acid position 317 of SEQ ID NO:2
(c) homozygosity or heterozygosity for the adenine at the nucleotide position corresponding to nucleotide position 951 of SEQ ID NO:1 or for the isoleucine at the amino acid position corresponding to amino acid position 317 of SEQ ID NO:2; and
(d) homozygosity for a guanine at a nucleotide position corresponding to nucleotide position 951 of SEQ ID NO:1 or for a methionine at an amino acid position corresponding to amino acid position 317 of SEQ ID NO:2.

39. The method of claim 37, wherein the clinical trial is selected from the group consisting of:
(a) a clinical trial for assessing the efficacy of a GPR109A receptor agonist for treating or preventing a lipid disorder without or with less of a condition associated with stimulation of MAP kinase activity by niacin or a niacin analog;
(b) a clinical trial for assessing the efficacy of a compound in ameliorating a condition associated with stimulation of MAP kinase activity by niacin or the niacin analog; and
(c) a clinical trial for assessing the efficacy of a compound for treating or preventing schizophrenia.

40. A method of determining a level of probability for a condition associated with stimulation of MAP kinase activity by niacin or a niacin analog for an individual having a GPR109A receptor zygosity;
said method comprising the steps of:
(a) identifying a clinical outcome for each of a plurality of patients in a clinical trial comprising a therapy, wherein the therapy comprises administration of an amount of niacin or a niacin analog, and wherein the clinical outcome is exhibiting or not exhibiting the condition associated with the stimulation of MAP kinase activity by niacin or the niacin analog;
(b) obtaining or identifying the GPR109A receptor zygosity for each of said plurality of patients in the clinical trial,
(c) associating the clinical outcome and the GRP109A receptor zygosity for each of said plurality of patients; and
(d) analyzing the associated clinical outcomes and GPR109A receptor zygosities so as to allow assignment of a level of probability for the condition associated with stimulation of MAP kinase activity by niacin or the niacin analog for the individual having the GPR109A receptor zygosity.

41. The method of claim 40, wherein the GPR109A receptor zygosity is selected from the group consisting of:
(a) homozygous for an adenine at a nucleotide position corresponding to nucleotide position 951 of SEQ ID NO:1 or for an isoleucine at an amino acid position corresponding to amino acid position 317 of SEQ ID NO:2;
(b) heterozygous for an adenine at a nucleotide position corresponding to nucleotide position 951 of SEQ ID NO:1 or for an isoleucine at an amino acid position corresponding to amino acid position 317 of SEQ ID NO:2; and
(c) homozygous for an G at a nucleotide position corresponding to nucleotide position 951 of SEQ ID NO:1 or for a methionine at an amino acid position corresponding to amino acid position 317 of SEQ ID NO:2.

42. The method of claim 40, wherein said analyzing comprises the steps of:
(a) segmenting or not segmenting the clinical outcomes on the basis of the GPR109A receptor zygosity so as to thereby make a segmented group and an unsegmented group; and
(b) comparing the clinical outcomes for the segmented group with the clinical outcomes for the unsegmented group.

43. A method of determining a level of probability for an individual for a condition associated with stimulation of MAP kinase activity by niacin or a niacin analog, said method comprising the steps of:
(a) obtaining a GPR109A receptor nucleic acid sequence or a GPR109A receptor amino acid sequence for the individual;
(b) identifying within said GPR109A receptor nucleic acid sequence a nucleotide polymorphism compared to SEQ ID NO:1, or within said GPR109A receptor amino acid sequence an amino acid polymorphism compared to SEQ ID NO:2; and
(c) assessing the ability of said GPR109A receptor nucleic acid sequence containing said nucleotide polymorphism, or GPR109A receptor amino acid sequence containing said amino acid polymorphism, to affect MAP kinase activation mediated by niacin, wherein a blunted MAP kinase activation compared to the MAP kinase activation of a GPR109A receptor containing SEQ ID NO:2 is associated with a decreased level of probability to the individual for the condition associated with stimulation of MAP kinase activity by niacin or the niacin analog.

44. The method of claim 1, 20, 26, 28, or 40, wherein the amount of niacin or the niacin analog is a therapeutically effective amount.

45. The method of claim 1, 11-29, 32, 36, 40, or 43, wherein the condition associated with a functional niacin receptor-mediated signal response or with the stimulation of MAP kinase activity by niacin or the niacin analog is cutaneous flushing.


SEQUENCE LISTING (12 sequences)

SEQ ID NO: 1 - DNA, 1092 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 2 - protein, 363 aa, Homo sapiens [sequence omitted]

SEQ ID NO: 3 - DNA, 32 bp, Homo sapiens
ctccactttgatcaactgctgcctccagagga

SEQ ID NO: 4 - DNA, 32 bp, Homo sapiens
tcctctggaggcagcagttgatcaaagtggag

SEQ ID NO: 5 - DNA, 1092 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaactgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 6 - protein, 363 aa, Homo sapiens
MNRHHLQDHFLEIDKKNCCVFRDDFIVKVLPPVLGLEFIFGLLGNGLALWIFCFHLKSWKSSRIFLFNLAVADFLLIICLPFLMDNYVRRWDWKFGDIPCRLMLFMLAMNRQGSIIFLTVVAVDRYFRVVHPHHALNKISNRTAAIISCLLWGITIGLTVHLLKKKMPIQNGGANLCSSFSICHTFQWHEAMFLLEFFLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINCCLQRKMTGEPDNNRSTSVELTGDPNKTRGAPEALMANSGEPWSPSYLGPTSP

SEQ ID NO: 7 - DNA, 31 bp, Homo sapiens
cctccagaggaagataacaggtgagccagat

SEQ ID NO: 8 - DNA, 31 bp, Homo sapiens
atctggctcacctgttatcttcctctggagg

SEQ ID NO: 9 - DNA, 1092 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagataacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 10 - protein, 363 aa, Homo sapiens [sequence omitted]

SEQ ID NO: 11 - DNA, 21 bp, Homo sapiens
gatgccgatccagaatggcgg

SEQ ID NO: 12 - DNA, 24 bp, Homo sapiens
ttcttggcatggttatttaaggag